(19) United States
(12) Patent Application Publication
(10) Pub. No.: US 2018/0117211 A1
(43) Pub. Date: May 3, 2018

(54) Perfusive Organ Hemostasis

(71) Applicant: Genzyme Corporation, Cambridge, MA (US)

(72) Inventors: Jean-Marie Vogel, Cambridge, MA (US); James A. Wilkie, Melrose, MA (US); Peter N. Madras, Newton, MA (US)

(21) Appl. No.: 15/621,704

(22) Filed: Jun. 13, 2017

Related U.S. Application Data
(63) Continuation of application No. 15/074,432, filed on Mar. 18, 2016, now abandoned.
Application No. 15/074,432 is a continuation of application No. 14/225,299, filed on Mar. 25, 2014, now abandoned.
Application No. 14/225,299 is a continuation of application No. 11/954,109, filed on Dec. 11, 2007, now abandoned.
(60) Provisional application No. 60/874,062, filed on Dec. 11, 2006.
(60) Provisional application No. 60/893,993, filed on Mar. 9, 2007.

(51) Int. Cl. A61L 24/04; A61B 17/00

(52) U.S. Cl. CPC A61L 24/046; A61L 2300/406; A61B 2017/12004; A61B 17/00491

(57) ABSTRACT

Disclosed are compositions, methods and kits to control bleeding through the use of an internal occluder based on polymeric solutions, including use of reverse thermosensitive polymers in nephron-sparing surgeries, which produces a completely bloodless surgical field, allowing speedy resection. In certain embodiments, after a certain amount of time, the flow gradually resumes, with no apparent adverse consequences to the kidney. In certain embodiments, return of blood flow may be accelerated by cooling the kidney. The compositions, methods and kits for perfusive organ hemostasis can also be used to simplify or to enable other organ surgeries or interventional procedures, including liver surgery, prostate surgery, brain surgery, surgery of the uterus, spleen surgery and any surgery on any highly vascularized organs.

### RELATED APPLICATIONS

This application is a continuation of U.S. application Ser. No. 15/074,432, filed Mar. 18, 2016, which is a continuation of U.S. application Ser. No. 14/225,299, filed Mar. 25, 2014, which is a continuation of U.S. application Ser. No. 11/954,109, filed Dec. 11, 2007, which claims the benefit of U.S. Application No. 60/874,062, filed Dec. 11, 2006; and U.S. Application No. 60/893,993, filed Mar. 9, 2007. The entire teachings of the above application(s) are incorporated herein by reference.

### BACKGROUND OF THE INVENTION

It is often medically desirable to limit reversibly blood flow in certain target anatomical sites. For example, in numerous surgeries, it is often desirable to occlude temporarily a blood vessel. Conventional hemostatic clamps such as the Fogarty clamp, the DeBakey “Atraugrip”, the Bulldog clamp or Pott's and Satinsky's peripheral vascular clamps are used extensively for occluding vessels. Although these conventional clamps are largely satisfactory in most instances wherein occlusion of a vessel is required, they have limited use in other applications which require hemostasis, such as sectioning large solid organs as in partial nephrectomy. The percentage of patients with renal cell carcinoma treated with partial nephrectomy has increased more than threefold from 3.7% (525 cases; 1988 to 1990) to 12.3% (4000 cases; 2000 to 2002). W. C. Huang, et al., “Chronic kidney disease after nephrectomy in patients with renal cortical tumors: a retrospective cohort study,” The Lancet Oncology 2006, 7(9), 735-740. Various other operations, such as hepatectomy, would also be facilitated by temporary blood flow interruption.

Nephron-Sparing Surgery—Partial Nephrectomy. Nephron-sparing surgery (NSS) in itself may prove to be suitable in a variety of contexts. For example, the curative management of renal cell carcinoma (RCC) remains surgical. Recent advances in preoperative staging, specifically modern imaging techniques, and improvements in surgical techniques have made partial nephrectomy an attractive alternative to radical nephrectomy in selected patients. NSS is more clearly indicated for cases in which a radical nephrectomy would render the patient anephric with a subsequent immediate need for dialysis. Synchronous bilateral tumors, tumors in a solitary kidney, or the presence of a poorly functional contralateral renal unit are generally absolute indications for NSS. The latter scenario could result from the concomitant presence of unilateral RCC and a contralateral kidney with disease processes (e.g., chronic pyelonephritis, renal arterial disease, calculus disease) or the presence of systemic diseases (e.g., diabetes). Partial nephrectomy may also be considered the treatment of choice for certain benign conditions and localized pathology of the kidney. A. C. Novick, “The role of nephron-sparing surgery for renal cell carcinoma in patients with a normal contralateral kidney,” Advan Urol 1996, 9,1. It allows for optimal surgical treatment and, at the same time, obviates over-treatment and nephron loss when possible and necessary. Examples of potentially more benign indications include traumatic irreversible injury to a localized portion of the kidney and removal of a benign renal tumor such as an oncocytoma, angiomyolipoma, or multilocular cyst. Other indications include an obstructed atrophied segment of a duplicated kidney, calculus disease of a renal segment with impaired drainage, and, rarely, renovascular hypertension with identifiable noncorrectible branch renal artery disease. R.G. Uzzo and A. C. Novick, “Nephron sparing surgery for renal tumors: indications, techniques and outcomes,” J. of Urol. 2001, 166, 6-18.

The clinical utility of NSS for RCC is revealed when several factors are considered. First, RCC usually does not become symptomatic until late in its course. Lesions detected incidentally tend to be smaller and of lesser grade, and thus more amenable to conservative surgery. The value of NSS is realized further when one considers the unreliability of current imaging studies in distinguishing between malignant and benign tumors of the kidney. Also, the natural history and malignant potential of small RCC is not well understood. Although observation could be a viable option in elderly patients with high comorbidities, NSS allows for curative surgery and elimination of uncertainty in the average patient with acceptable expected longevity. The goals of conservative resection of RCC are complete local surgical removal of the malignancy and preservation of adequate renal function. This is a delicate balance, which makes renal-preserving surgery, at times, both challenging and controversial. R. G. Uzzo and A. C. Novick, “Nephron sparing surgery for renal tumors: indications, techniques and outcomes,” J. of Urol. 2001, 166, 6-18.

Several surgical techniques are available for performing nephron-sparing surgery in patients with renal tumors. The five main surgical processes include enucleation of tissue, polar segmental nephrectomy, wedge resection, major transverse resection, and extracorporeal partial nephrectomy followed by renal autotransplantation.

All of these techniques require steady vascular control and thorough hemostasis, avoidance of renal ischemia, complete tumor removal with free margins, and efficient closure of the intrarenal collecting system. Finally, an adequate postoperative renal function must be maintained since a functioning renal remnant of at least twenty percent (20%) of one kidney is necessary to avoid end-stage renal failure. However, it is important not to compromise the extent of the surgical procedure to preserve renal function at the expense of an incomplete resection. Postoperative renal insufficiency typically results from a combination of intraoperative ischemia and loss of functioning renal parenchyma. The extent of renal insufficiency varies, and its degree is reflected by the increase of retention parameters such as creatinine, blood urea, and potassium. Severe renal insufficiency may require temporary dialysis. If the compensatory hypertrophy of the remnant kidney tissue cannot compensate for the loss of renal function, a permanent insufficiency requiring permanent dialysis may result. The main steps of conventional partial nephrectomy include initiating diuresis with intravenous mannitol and a loop diuretic (e.g., furosemid) intraoperatively, with generous hydration before any interruption in the renal circulation. Mannitol is infused before anticipated renal occlusion. This agent not only induces osmotic diurisis but also is a free radical scavenger that can minimize ischemic insult from arterial clamping and the ultimate risk of postoperative acute tubular necrosis.

In partial nephrectomy, an incision is made of either the bilateral subcostal or thoracoabdominal type. After opening the abdomen, the colon is moved to expose the kidney. The renal artery is temporarily clamped to reduce bleeding. Typically, the renal artery is occluded with an atraumatic vascular Bulldog clamp. The renal vein may remain non-occluded since retrograde profusion of the kidney might minimize the chance for acute tubular necrosis postoperatively. The kidney is dissected from the surrounding tissue from outside the renal fascia. The tumor is removed with a margin of normal tissue. The calyxes and renal pelvis that have been cut through are carefully closed with sutures. The cut end of the kidney is covered with fat, fascia or peritoneum. The clamp on the renal artery is removed and all bleeding is controlled prior to the incision being closed.

Issues with Current Hemostatic Approaches. One of the main drawbacks associated with the conventional partial nephrectomy method is that clamping of the renal artery causes ischemia of the whole kidney. Although the ischemia is typically transient it may nevertheless lead to renal insufficiency if the arterial clamp time is extended. Attention to intraoperative measures to decrease the possibility of this complication, such as hydrating preoperatively, correcting electrolyte abnormalities, using mannitol and potentially using surface hypothermia may prove to be insufficient in some unfortunate instances. Some unfortunate patients may hence need renal replacement therapy, for example hemodialysis.

The technical literature reflects a significant effort in the medical research community directed to the development of an understanding of the damage observed in reperfused ischemic tissue. In fact, researchers have found that significant tissue damage resulting after a period of tissue ischemia, followed by reperfusion, occurs not only during the period of circulatory arrest, but during the period of reperfusion. Indeed, a relatively large portion of the total injuries seen after five to sixty minute periods of circulatory arrest may actually develop during the reperfusion stage. Such tissue damage is known as reperfusion injury.

Clamping and subsequent release of the renal artery may hence potentially lead not only to ischemia injury but also to reperfusion injuries. Some authorities believe that irreversible renal lesions occur when total renal ischemia resulting from clamping of the renal artery exceeds twenty minutes.

Another troublesome and more common intraoperative complication of the conventional partial nephrectomy method is excessive bleeding. Easy access to the renal hilum, provided by early identification and isolation of the renal artery, provides additional safety of prompt arterial occlusion when excessive bleeding precludes a clear surgical field and adequate visualization. However, in some situations this may prove to be insufficient, potentially leading to the need for embolization or re-exploration in the case of severe intractable bleeding.

In an attempt to circumvent the mentioned disadvantages associated with clamping of the renal artery during conventional nephron sparing or partial nephrectomy, some surgeons have attempted to clamp a segment of tissue surrounding the mass to be excised. The goal is to use a renal tourniquet in order to localize the ischemia to the tissue which is to be removed and its immediate periphery. Although reducing ischemia to the remainder of the kidney is theoretically appealing, attempts at clamping tumor-adjacent kidney tissue instead of the renal artery during partial nephrectomy have proven to be unsuccessful due to unreliable hemostasis during surgery.

Problems associated with attempts at clamping kidney tissue instead of the kidney arteries may be, at least partially, imputable to the use of conventional vascular clamps to perform the tissue clamping operation. As is well known, conventional vascular clamps typically include a pair of pivoting arms with a clamping jaw rigidly attached to a distal end of each pivoting arm. The process of clamping generates loci of high pressure far in excess of the pressure in the blood vessel itself. Conventional clamps such as the Fogarty clamp, the DeBakey “Atraugrip”, the Bulldog clamp or Pods and Satinsky's peripheral vascular clamps exert relatively high pressures, in some cases up to nine bars on clamped blood vessels. One of the drawbacks associated with conventional vascular clamps when used for clamping tissue is that the applied pressure is distributed in a non-uniform manner at the interface between the clamping jaw and the tissue. Indeed, the conventional clamping jaws (typically being of the scissor type) create a gradient of applied pressure along the clamping jaws with the higher pressure being located adjacent to the proximal end near the hinge. This is of particular concern in sick patients who are more likely to have calcified or stenosed renal arteries. R. D. Safian, S. C. Textor, “Renal-Artery Stenosis,” N Engl J Med 2001, 344(6), 431-442.

This leads to excessively high pressures in some areas, potentially leading to undue injury of adjacent tissue and to unsuitable hemostasis from insufficient pressure at distal locations. In view of the fact that systemic blood pressure is at least one order of magnitude lower than pressure applied to the tissue by conventional clamps, it becomes evident that suitable hemostasis could be achieved at far lower pressures than those exerted adjacent to the proximal end of the jaws. Furthermore, the configuration of most conventional vascular clamps has further proven to be unsuitable since it prevents insertion of body tissues of various configurations in size.

Minimally Invasive Techniques. There is a clear trend in Urology towards robotic and laparoscopic minimally invasive techniques. Adequate hemostasis of the renal surface is essential for laparoscopic nephron-sparing surgery because uncontrolled bleeding may cause significant complications and even conversion to laparotomy. As a general rule, hemostasis during laparoscopic surgery plays a pivotal role and aims to primarily prevent bleeding or at least provide early vascular and bleeding control. Because even minor bleeding may cause impaired vision due to significant light absorption by dark blood staining of the adjacent tissues, thereby compromising the advantages of the magnified vision offered by the laparoscope, a wide variety of tissue sealants have been adapted or developed for laparoscopic surgery and purposed especially for nephron-sparing surgery.

Sealants. The use of tissue sealants in partial nephrectomy allows for a fast parenchyma, vascular, and collecting system repair and reduces not only the overall operative time but particularly the warm ischemia time. Therefore, the negative impact on the renal function of temporary vascular clamping can be reduced, of pivotal importance especially in patients with a solitary kidney or with impaired renal function.

Among all the hemostatic tools available, “glues,” or tissue sealants, are the only adequate alternatives for bleeding control of the cut renal surface, and their use as unique hemostatic agents is adequate in cases of small peripheral lesions. Sealants can be divided into two categories: non-biologic and biologic. Among the non-biologic glues, the best known is probably 2-octyl-cyanoacrylate. Primarily used for skin closure, it creates a fixed and watertight lining over the cut renal parenchyma within 2-3 minutes. To work, it requires a bloodless field, making preventive hemostasis with hilar clamping essential. Particular care must be paid to avoid accidental contact with the surrounding tissues during its positioning to avoid gluing of other structures, such as the renal pedicle or ureter. All the biologic sealants include thrombin or fibrinogen or both. They are the terminal product of the clotting process and their use determines a fibrin matrix over the site of apposition. Probably the best known biologic sealants are fibrin glues (Tissuecol, Crosseal, Tisseel). They combine, in two separate syringes injected simultaneously in the site of action, human thrombin and fibrinogen and create a veil that seals small vascular lesions and favors hemostatic processes. Gelatin matrix hemostatic sealant (FloSeal) is a more recent solution widely used as a hemostatic sealant. It is a combination of a bovine gelatin-based matrix with a bovine-derived concentrated thrombin component. This viscous collagen matrix requires active bleeding to be activated and work, promoting coagulation and hemostasis at the site of bleeding; it requires 1-2 minutes of delicate compression on the cut edge of the parenchyma after its apposition.

### SUMMARY OF THE INVENTION

The inventive compositions, methods and kits provided herein serve to control bleeding through the use of an internal occluder based on polymeric solutions. For example, herein is disclosed the use of reverse thermosensitive polymers in nephron-sparing surgeries. In one embodiment of this approach, the renal artery leading to a diseased kidney is infused with a transient reverse thermosensitive gel. This has been shown to lead to cessation of blood flow within the renal parenchyma. It appears that, with the appropriate injection rate, the polymer flows downstream to occlude small, intra-renal vessels on both the arterial and venous sides of the circulation. Remarkably, this produces a completely bloodless surgical field, allowing speedy resection. In certain embodiments, after a certain amount of time, the flow gradually resumes, with no apparent adverse consequences to the kidney. In certain embodiments, return of blood flow may be accelerated by cooling the kidney.

In other embodiments, the inventive perfusive organ hemostasis just described can be used to simplify or to enable other organ surgeries or interventional procedures. In other embodiments, the inventive perfusive organ hemostasis just described can be used to simplify or to enable liver surgery, prostate surgery, brain surgery, surgery of the uterus, spleen surgery and any surgery on any highly vascularized organs. In certain embodiments the compositions, methods and kits described for perfusive organ hemostasis can be used for intervention on scleroses arteries, intervention on calcified vessels, as well as many other surgical and interventional uses.

### BRIEF DESCRIPTION OF THE DRAWINGS

FIG. 1A-1C depict occlusion using internal vessel occluder during partial nephrectomy in pigs at 15 minutes (A), 30 minutes (B), and 50 minutes (C). Fifteen minutes after injection the kidney is completely avascular and no bleeding is observed from the cut edges. Thirty minutes after injection, kidney perfusion is returning and not a drop of blood is observed. Fifty minutes after injection, the kidney has returned to normal; this was verified by pathology reports.

FIG. 2 depicts a graph showing the amount of blood collected in 15 minutes during coronary anastomosis in four pigs treated either with tape or a combination of tape and a polymer composition of the invention (i.e., 20% solution of poloxamer 407 in saline).

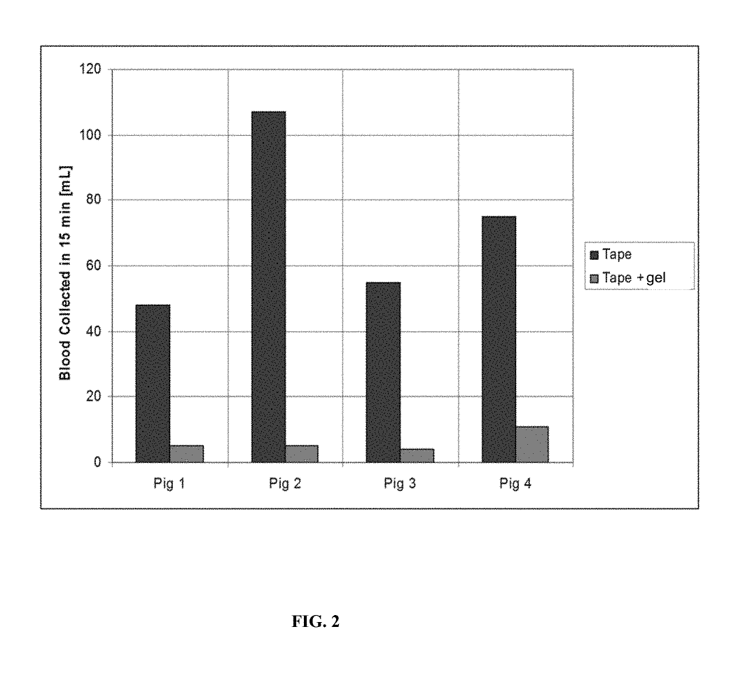

FIGS. 3A-3D depicts the use of the polymers of the invention (e.g., 20% solution of poloxamer 407) in a bypass surgery. The polymeric material is easy to inject (A), creating a pristine bloodless surgical field (B), allowing easy sewing (C), and disappears after use (D).

FIG. 4 depicts a graph of viscosity as a function of temperature for various solutions of purified poloxamer 407.

FIG. 5 depicts a table (Table 1) showing the purification of poloxamer 407; and a table (Table 2) showing the gelation temperature of selected reverse thermosensitive polymers in saline. In Table 1, a “*” indicates a viscosity of a 25% solution measured at 30° C. using a cone and plate viscometer.

### DETAILED DESCRIPTION OF THE INVENTION

Surgically removing only the morbid part of a diseased kidney instead of removing the entire kidney is beneficial for overall long-term kidney function. The technical hurdles limiting the adoption of this approach are the ability to control bleeding during the surgery and the need to reduce warm ischemia time. A bloodless field is also vital to the success of laparoscopic minimally invasive techniques. Herein is provided a technical solution, perfusive organ hemostasis, to control bleeding during the procedure by filling the kidney with a biocompatible gel which completely prevents bleeding and makes the surgery easier, potentially reducing the duration of the procedure. In other embodiments, the inventive perfusive organ hemostasis described can be used to simplify or to enable other organ surgeries or interventional procedures. In certain embodiments, the inventive perfusive organ hemostasis described can be used to simplify or to enable liver surgery, prostate surgery, brain surgery, surgery of the uterus, spleen surgery, as well as any surgery on any highly vascularized organs. In certain embodiments, the compositions, methods and kits described for perfusive organ hemostasis can be used for intervention on sclerotic arteries, intervention on calcified vessels, as well as many other surgical and interventional uses.

One aspect of the invention relates to compositions, methods and kits for controlling bleeding through the use of an internal occluder based on polymeric solutions. In certain embodiments, these solutions are reverse thermosensitive polymeric solutions. At low temperatures a reverse thermosensitive polymer solution is a liquid and as the temperature increases to body temperature, its viscosity increases several orders of magnitude to the consistency of a hard gel, leading to occlusion of the vessels. Cooling the occlusion site lowers the viscosity back to that of the liquid, dissolving the gel in blood and reestablishing blood flow. By applying the vessel occlusion internally, calcified arteries may be gently occluded, stopping the arterial and venous blood supply to an organ. Remarkably, this occlusion will prevent bleeding and provide a surgeon with a bloodless surgical field. The bloodless surgical field, in turn, should reduce the operating time required for a surgery. In certain surgeries, the operating time could be reduced to such an extent that warm ischemia time would be drastically reduced.

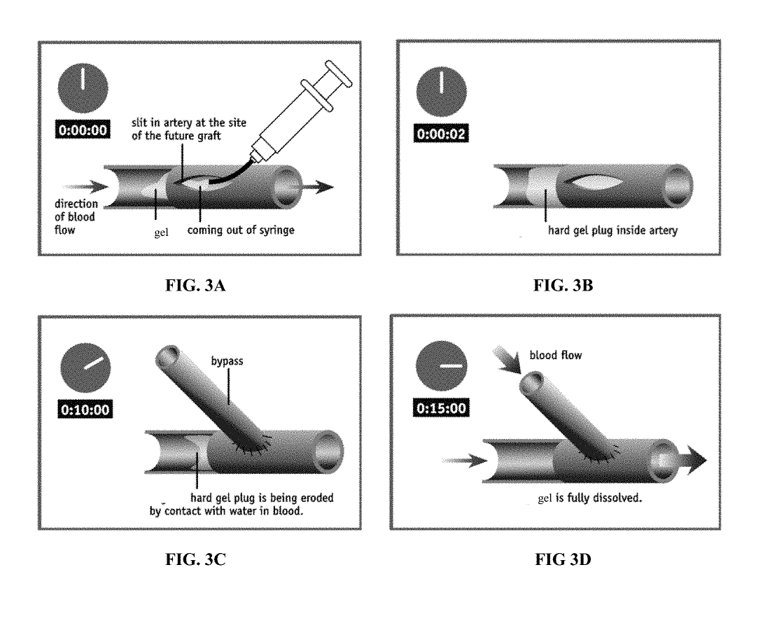

As mentioned above, a method wherein a biocompatible gel is used to fill and occlude a blood vessel has been developed. In certain embodiments, the gel's working principle is based on the reverse thermosensitive properties of the polymer. At low temperatures, the polymeric solution is a liquid. As the temperature increases to body temperature, the viscosity of the solution rapidly increases several orders of magnitude to the consistency of a hard gel. Cooling the occlusion site, simply by the application of ice, lowers the viscosity back to that of a liquid, dissolving the gel in blood and reestablishing blood flow. The gel has been developed for applications in anastomosis such as Off-Pump Bypass surgery (OPCAB), hemodialysis access, and tibial anastomosis. See FIG. 3A-D. It has been shown to work very gently, neither compromising nor changing the biochemical make-up the arterial wall, as evidenced by measurements of the microvascular reactivity after filling and opening up the blood vessel. M. Boodwhani, W. E. Cohn, J. Feng, B. Ramlavi, S. Mieno, A. Schwarz, and F. W. Sellke, “Safety and Efficacy of a Novel Gel for Vascular Occlusion in Off-Pump Surgery,” Ann Thorac Surg 2005, 80, 2333-7.

As proposed herein, the transient gels of the invention offer great advantages for intraoperative hemostasis, by allowing a solution to perfuse into and solidify in the both the arterial and venous sides of the organ's circulation, thereby completely or substantially occluding the blood supply of the organ. For example, herein is disclosed the use of reverse thermosensitive polymers in nephron-sparing surgery. In certain embodiments, the renal artery leading to the diseased kidney is infused with the reverse thermosensitive gel. This has been shown to lead to cessation of blood flow within the renal parenchyma. It appears that, with the appropriate injection rate, the polymer flows downstream to occlude small, intra-renal vessels on both the arterial and venous sides of the circulation. Remarkably, this produces a completely bloodless surgical field, allowing speedy resection. Under certain conditions, after about 20 minutes the flow gradually resumes, with no apparent adverse consequences to the kidney. In certain embodiments, return of blood flow may then be accelerated, if necessary, by cooling the kidney.

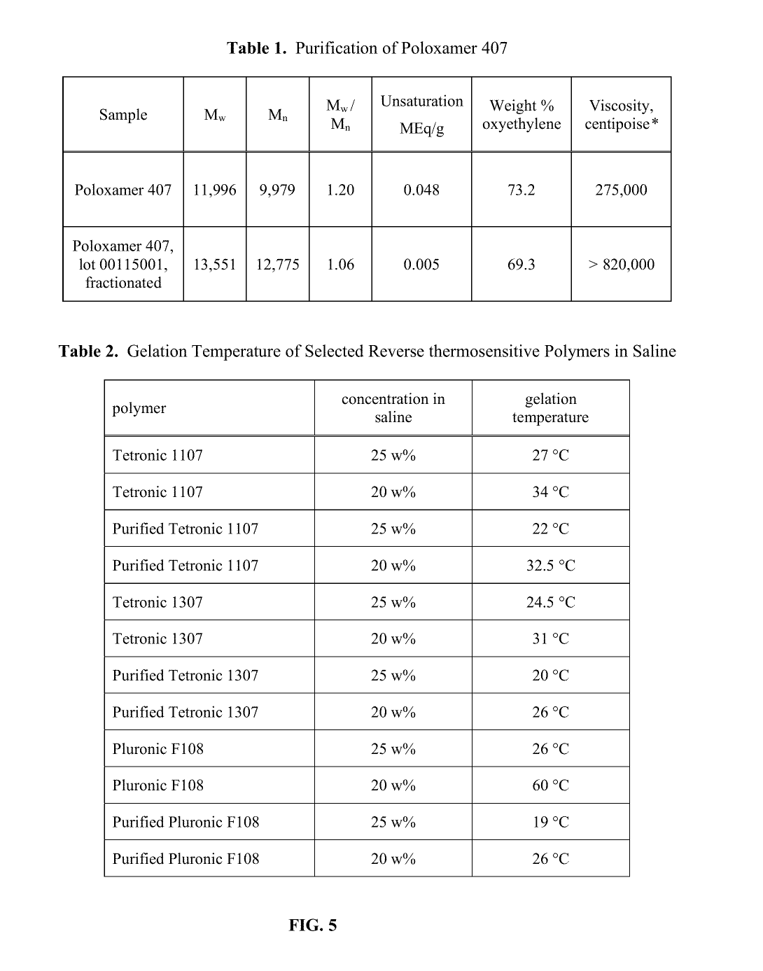

As described in greater detail below, initial short-term in-vivo experiments indicate the feasibility of this approach. However, the polymer used in some of the Exemplification has a lower than optimal transition temperature for a solid organ. In a solid organ, such as the kidney, temperatures during the surgery may be higher than in exposed arteries where the company has greater experimental experience. A polymer solution with a higher transition temperature may be superior to the present formulation for solid organ applications. Several such higher transition temperature polymer solutions are disclosed herein (see FIG. 5, Table 2). In certain embodiments, the rate/volume of injection may be used to control expected downstream ischemic time.

Additionally, an injection system that does not unduly increase the procedural time by requiring a long dissection of the artery or requiring additional surgery to repair the artery at the injection site is claimed herein. In certain embodiments, in order to adequately occlude a kidney for partial nephrectomy, for example, the polymeric gel must flow into the renal artery and then into the vascular structure of the kidney. The gel may be injected into either the renal artery or in the aorta immediately adjacent to the renal artery. After application the surgeon must close the injection site to prevent post operative bleeding. However, patients with diseased kidneys typically have calcified arteries, so a smaller puncture site is desirable. If the aorta is chosen as the injection site, the tip of the injector must enter the renal artery to direct the flow of gel into the kidney. A surgeon must weigh certain options when choosing an injection site: 1) the degree of difficulty in obtaining access to the renal artery, 2) the degree of difficulty in successfully cannulating a calcified vessel and subsequently closing the puncture site, and 3) in the event that the aorta is preferable, the ease with which the injector tip can be directed to the renal artery. One must also seek to minimize any time or effort spent to dissect a vessel prior to injection. Further, it may be advantageous to leave the injection system in place during the partial nephrectomy in the event that an additional dose of the gel is needed, and as a conduit to inject saline solution if needed for earlier dissolution of the gel.

Definitions. For convenience, before further description of the present invention, certain terms employed in the specification, examples, and appended claims are collected here. These definitions should be read in light of the remainder of the disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

When used with respect to a therapeutic agent or other material, the term “sustained release” is art-recognized. For example, a subject composition which releases a substance over time may exhibit sustained release characteristics, in contrast to a bolus type administration in which the entire amount of the substance is made biologically available at one time.

The term “poloxamer” denotes a symmetrical block copolymer, consisting of a core of PPG polyoxyethylated to both its terminal hydroxyl groups, i.e., conforming to the interchangable generic formula (PEG)_{X}-(PPG)_{Y}-(PEG)_{X} and (PEO)_{X}-(PPO)_{Y}-(PEO)_{X}. Each poloxamer name ends with an arbitrary code number, which is related to the average numerical values of the respective monomer units denoted by X and Y.

The term “poloxamine” denotes a polyalkoxylated symmetrical block copolymer of ethylene diamine conforming to the general type [(PEG)_{X}-(PPG)_{Y}]₂-NCH₂CH₂N-[(PPG)_{Y}-(PEG)_{X}]₂. Each Poloxamine name is followed by an arbitrary code number, which is related to the average numerical values of the respective monomer units denoted by X and Y.

The term “reverse thermosensitive polymer” as used herein refers to a polymer that is soluble in water at ambient temperature, but at least partially phase-separates out of water at physiological temperature. Reverse thermosensitive polymers include, for example, poloxamer 407, poloxamer 188, Pluronic® F127, Pluronic® F68, poly(N-isopropylacrylamide), poly(methyl vinyl ether), poly(N-vinylcaprolactam); and certain poly(organophosphazenes). See: B. H. Lee, et al. “Synthesis and Characterization of Thermosensitive Poly(organophosphazenes)with Methoxy-Poly(ethylene glycol) and Alkylamines as Side Groups,” Bull. Korean Chem. Soc. 2002, 23, 549-554.

The terms “reversibly gelling” and “reverse thermosensitive” refer to the property of a polymer wherein gelation takes place upon an increase in temperature, rather than a decrease in temperature.

The term “transition temperature” refers to the temperature or temperature range at which gelation of an reverse thermosensitive polymer occurs.

The term “degradable”, as used herein, refers to having the property of breaking down or degrading under certain conditions, e.g., by dissolution.

The phrase “polydispersity index” refers to the ratio of the “weight average molecular weight” to the “number average molecular weight” for a particular polymer; it reflects the distribution of individual molecular weights in a polymer sample.

The phrase “weight average molecular weight” refers to a particular measure of the molecular weight of a polymer. The weight average molecular weight is calculated as follows: determine the molecular weight of a number of polymer molecules; add the squares of these weights; and then divide by the total weight of the molecules.

The phrase “number average molecular weight” refers to a particular measure of the molecular weight of a polymer. The number average molecular weight is the common average of the molecular weights of the individual polymer molecules. It is determined by measuring the molecular weight of n polymer molecules, summing the weights, and dividing by n.

The term “biocompatible”, as used herein, refers to having the property of being biologically compatible by not producing a toxic, injurious, or immunological response in living tissue.

As used herein “cold-packs” are two containers containing chemicals separated by a frangible seal. When the seal is broken, as the contents from the separate containers begin to react, energy is absorbed from the surroundings creating a cooling effect. An example of chemicals which can be mixed in a cold pack are ammonium nitrate and water. In certain embodiments the cold pack has two sealed bags, one inside the other. The outer bag is made of thick strong plastic. It contains a ammonium nitrate and the second plastic bag. The second (inner) bag is made of a thin weak plastic and contains water. When the bag is squeezed the inner bag breaks and the water mixes with the powder creating the cooling effect.

The term “hemostasis” refers to the stoppage of blood flow through a blood vessel or organ of the body. Hemostasis generally refers to the arrest of bleeding, whether it be by normal vasoconstriction (the vessel walls closing temporarily), by an abnormal obstruction (such as a plaque) or by coagulation or surgical means (such as ligation). As used herein, hemostasis is achieved by using a transient gel to create an obstruction.

Contemplated equivalents of the polymers, subunits and other compositions described above include such materials which otherwise correspond thereto, and which have the same general properties thereof (e.g., biocompatible), wherein one or more simple variations of substituents are made which do not adversely affect the efficacy of such molecule to achieve its intended purpose. In general, the compounds of the present invention may be prepared by, for example, described below, or by modifications thereof, using readily available starting materials, reagents and conventional synthesis procedures. In these reactions, it is also possible to make use of variants which are in themselves known, but are not mentioned here.

Selected Applications. One aspect of the invention relates to compositions, methods and kits for partial nephrectomy which prevents renal surface bleeding and reduces warm ischemia time during the surgery, leading to improved patient outcomes. Although partial nephrectomy is beneficial to the patient due to its kidney sparing effect, currently only about 12% of all nephrectomies are performed as partial nephrectomies. This is partially due to the technical difficulties encountered during the procedure. B. A. Kletscher, et al., “Nephron-Sparing laparoscopic surgery: techniques to control the renal pedicle and manage parenchymal bleeding,” J Endourol 1995, 9, 23; and W. C. Huang, et al., “Chronic kidney disease after nephrectomy in patients with renal cortical tumours: a retrospective cohort study,” The Lancet Oncology 2006, 7(9), 735-740. While there have been numerous attempts published in the literature to control bleeding of the renal surface, all attempting to control it by applying agents or energy to the renal surface, most if not all of these nephron-sparing surgical methods suffer from at least one of two identified technical problems: the requirement to reduce warm ischemia times to preferentially less than 20 minutes; and the requirement for adequate hemostasis of the renal surface. The latter requirement for a bloodless field is amplified by the trend in urologic surgery towards robotic and minimally invasive techniques where visibility can be severely limited by even small volumes of blood. R. G. Uzzo, A. C. Novick, “Nephron sparing surgery for renal tumors: indications, techniques and outcomes,” J. of Urol. 2001, 166, 6-18.

In certain embodiments, the methods of the present invention combine the transient gel with the use of robotic and laparoscopic techniques in order to reduce blood loss and operative time with these minimally invasive techniques.

The perfusive organ hemostasis of the invention can also in other embodiments be used to simplify or to enable other organ surgeries or interventional procedures, such as liver surgery (e.g., a partial hepatectomy), prostate surgery (e.g., a full or partial prostatectomy), brain surgery, surgery of the uterus, spleen surgery and any surgery on any highly vascularized organs. In certain embodiments the compositions, methods and kits described for perfusive organ hemostasis can be used for intervention on scleroses arteries; intervention on calcified vessels; as well as for many other surgical and interventional uses.

Transient Gels of the Invention. In certain embodiments, the perfusive organ hemostasis of the invention may be accomplished by the use of polymers that form a gel inside the body and then dissolve or are dissolved, such as other reverse thermosensitive polymers and any polymer solution or combination of polymers that form a gel inside the body, being under the effect of temperature, pH, pressure, or as a result of a chemical or biological reaction. In other embodiment, the transient gels used in a method of the invention are crosslinkable polymers. In certain embodiments, the transient gels can be generated in situ. In certain embodiments, the transient gels can be non-tissue adhesive.

In certain embodiments, two solutions, a polymer solution and a crosslinker solution, are injected separately (e.g., through a dual lumen catheter) into a biological lumen wherein they gel, forming a transient gel. Said polymer solution may comprise an anionic polymer, a cationic polymer or a non-ionically crosslinkable polymer. Such polymers may comprise one or more of the following: alginic acid, sodium alginate, potassium alginate, sodium gellan, potassium gellan, carboxy methyl cellulose, hyaluronic acid, and polyvinyl alcohol. The cross-linking of the polymer to form a polymer gel may be achieved with anionic crosslinking ions, cationic crosslinking ions, or non-ionic crosslinking agents. Crosslinking agents include, but are not limited to, one or more of the following: phosphate, citrate, borate, succinate, maleate, adipate, oxalate, calcium, magnesium, barium and strontium. Exemplary pairings of polymers and crosslinkers include anionic polymer monomers with cations, such as, for example, alginates with calcium, barium or magnesium; gellans with calcium, magnesium or barium; or hyaluronic acid with calcium. An example of an exemplary pairing of a non-ionic polymer with a chemical crosslinking agent is a polyvinyl alcohol with borate (at a slightly alkaline pH).

In general, the polymers used in the methods of the invention, which become a gel at or about body temperature, can be administered in a liquid form. In certain embodiments, the polymer composition of the invention may be a flexible or flowable material. By “flowable” is meant the ability to assume, over time, the shape of the space containing it at body temperature. This characteristic includes, for example, liquid compositions that are suitable for: injection with a manually operated syringe fitted with, for example, a needle; or delivery through a catheter. Also encompassed by the term “flowable” are highly viscous, gel-like materials at room temperature that may be delivered to the desired site by pouring, squeezing from a tube, or being injected with any one of the commercially available power injection devices that provide injection pressures greater than would be exerted by manual means alone. When the polymer used is itself flowable, the polymer composition of the invention, even when viscous, need not include a biocompatible solvent to be flowable, although trace or residual amounts of biocompatible solvents may be present.

In addition, in certain embodiments, the transient gel of the invention may be aqueous solution of one or more reverse thermosensitive polymers. These polymer solutions are liquids below body temperature and gel at about body temperature. In certain embodiments, the polymer solution is prepared external of the body, i.e., at a temperature below body temperature. The polymer solution may be further chilled to prolong the time the gel stays in the liquid form upon introduction into the body. A preferred temperature is about 10° C. below the gelation temperature of the polymer solution. In certain embodiments, the transient gel used in connection with the methods of the invention may comprise a block copolymer with inverse thermal gelation properties. The block copolymer can further comprise a polyoxyethylene-polyoxypropylene block copolymer, such as a biodegradable, biocompatible copolymer of polyethylene oxide and polypropylene oxide. Also, the reverse thermosensitive polymer can include one or more additives; for example, therapeutic agents may be added to the reverse thermosensitive polymers.

In certain embodiments, the block copolymers have molecular weights ranging from about 2,000 to about 1,000,000 Daltons, more particularly at least about 10,000 Daltons, and even more specifically at least about 25,000 Daltons or even at least about 50,000 Daltons. In certain embodiment, the block copolymers have a molecular weight between about 5,000 Daltons and about 30,000 Daltons. In certain embodiments, the molecular weight of the reverse thermosensitive polymer may be between about 1,000 and about 50,000 Daltons, or between about 5,000 and about 35,000 Daltons. In other embodiments, the molecular weight of a suitable reverse thermosensitive polymer (such as a poloxamer or poloxamine) may be, for example, between about 5,000 and about 25,000 Daltons, or between about 7,000 and about 20,000 Daltons. Number-average molecular weight (Mₙ) may also vary, but will generally fall in the range of about 1,000 to about 400,000 Daltons, in some embodiments from about 1,000 to about 100,000 Daltons and, in other embodiments, from about 1,000 to about 70,000 Daltons. In certain embodiments, Mₙ varies between about 5,000 and about 300,000 Daltons.

In certain embodiments, the polymer is in an aqueous solution. For example, typical aqueous solutions contain about 5% to about 30% polymer, preferably about 10% to about 25%. The pH of the reverse thermosensitive polymer formulation administered to a mammal is, generally, about 6.0 to about 7.8, which are suitable pH levels for injection into the mammalian body. The pH level may be adjusted by any suitable acid or base, such as hydrochloric acid or sodium hydroxide.

In certain embodiments, the reverse thermosensitive polymers of the invention are poloxamers or poloxamines. Pluronic® polymers have unique surfactant abilities and extremely low toxicity and immunogenic responses. These products have low acute oral and dermal toxicity and low potential for causing irritation or sensitization, and the general chronic and sub-chronic toxicity is low. In fact, Pluronic® polymers are among a small number of surfactants that have been approved by the FDA for direct use in medical applications and as food additives. See: BASF (1990) Pluronic® & Tetronic® Surfactants, BASF Co., Mount Olive, N.J. Recently, several Pluronic® polymers have been found to enhance the therapeutic effect of drugs, and the gene transfer efficiency mediated by adenovirus. K. L. March, J. E. Madison, and B. C. Trapnell, “Pharmacokinetics of adenoviral vector-mediated gene delivery to vascular smooth muscle cells: modulation by poloxamer 407 and implication for cardiovascular gene therapy,” Hum Gene Therapy 1995, 6, 41-53.

Interestingly, poloxamers (or Pluronics), as nonionic surfactants, are widely used in diverse industrial applications. See, for example, Nonionic Surfactants: polyoxyalkylene block copolymers, Vol. 60. Nace V M, Dekker M (editors), New York, 1996. 280 pp. Their surfactant properties have been useful in detergency, dispersion, stabilization, foaming, and emulsification. A. Cabana, A. K. Abdellatif, and J. Juhasz, “Study of the gelation process of polyethylene oxide. polypropylene oxide-polyethylene oxide copolymer (poloxamer 407) aqueous solutions.” Journal of Colloid and Interface Science 1997, 190, 307-312. Certain poloxamines, e.g., poloxamine 1307 and 1107, also display inverse thermosensitivity.

Importantly, several members of this class of polymer, poloxamer 188, poloxamer 407, poloxamer 338, poloxamine 1107 and poloxamine 1307 show inverse thermosensitivity within the physiological temperature range. Y. Qiu, and K. Park, “Environment-sensitive hydrogels for drug delivery.” Adv Drug Deliv Rev 2001, 53(3), 321-339; and E. S. Ron, and L. E. Bromberg, “Temperature-responsive gels and thermogelling polymer matrices for protein and peptide delivery,” Adv Drug Deliv Rev 1998, 31(3), 197-221. In other words, these polymers are members of a class that are soluble in aqueous solutions at low temperature, but gel at higher temperatures. Poloxamer 407 is a biocompatible polyoxypropylene-polyoxyethylene block copolymer having an average molecular weight of about 12,500 and a polyoxypropylene fraction of about 30%; poloxamer 188 has an average molecular weight of about 8400 and a polyoxypropylene fraction of about 20%; poloxamer 338 has an average molecular weight of about 14,600 and a polyoxypropylene fraction of about 20%; poloxamine 1107 has an average molecular weight of about 14,000, poloxamine 1307 has an average molecular weight of about 18,000. Polymers of this type are also referred to as reversibly gelling because their viscosity increases and decreases with an increase and decrease in temperature, respectively. Such reversibly gelling systems are useful wherever it is desirable to handle a material in a fluid state, but performance is preferably in a gelled or more viscous state. As noted above, certain poly(ethyleneoxide)/poly(propyleneoxide) block copolymers have these properties; they are available commercially as Pluronic® poloxamers and Tetronic® poloxamines (BASF, Ludwigshafen, Germany) and generically known as poloxamers and poloxamines, respectively. See U.S. Pat. Nos. 4,188,373, 4,478,822 and 4,474,751; all of which are hereby incorporated by reference.

The average molecular weights of commercially available poloxamers and poloxamines range from about 1,000 to greater than 16,000 Daltons. Because the poloxamers are products of a sequential series of reactions, the molecular weights of the individual poloxamer molecules form a statistical distribution about the average molecular weight. In addition, commercially available poloxamers contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15% to about 50% of the total mass of the commercial polymer.

The reverse thermosensitive polymers may be purified using a process for the fractionation of water-soluble polymers, comprising the steps of dissolving a known amount of the polymer in water, adding a soluble extraction salt to the polymer solution, maintaining the solution at a constant optimal temperature for a period of time adequate for two distinct phases to appear, and separating physically the phases. Additionally, the phase containing the polymer fraction of the preferred molecular weight may be diluted to the original volume with water, extraction salt may be added to achieve the original concentration, and the separation process repeated as needed until a polymer having a narrower molecular weight distribution than the starting material and optimal physical characteristics can be recovered.

In certain embodiments, a purified poloxamer or poloxamine has a polydispersity index from about 1.5 to about 1.0. In certain embodiments, a purified poloxamer or poloxamine has a polydispersity index from about 1.2 to about 1.0.

The aforementioned process consists of forming an aqueous two-phase system composed of the polymer and an appropriate salt in water. In such a system, a soluble salt can be added to a single phase polymer-water system to induce phase separation to yield a high salt, low polymer bottom phase, and a low salt, high polymer upper phase. Lower molecular weight polymers partition preferentially into the high salt, low polymer phase. Polymers that can be fractionated using this process include polyethers, glycols such as poly(ethylene glycol) and poly(ethylene oxide)s, polyoxyalkylene block copolymers such as poloxamers, poloxamines, and polyoxypropylene/polyoxybutylene copolymers, and other polyols, such as polyvinyl alcohol. The average molecular weight of these polymers may range from about 800 to greater than 100,000 Daltons. See U.S. Pat. No. 6,761,824 (hereby incorporated by reference). The aforementioned purification process inherently exploits the differences in size and polarity, and therefore solubility, among the poloxamer molecules, the poly(oxyethylene) homopolymer and the poly(oxyethylene)/poly(oxypropylene) diblock byproducts. The polar fraction of the poloxamer, which generally includes the lower molecular weight fraction and the byproducts, is removed allowing the higher molecular weight fraction of poloxamer to be recovered. The larger molecular weight poloxamer recovered by this method has physical characteristics substantially different from the starting material or commercially available poloxamer including a higher average molecular weight, lower polydispersity and a higher viscosity in aqueous solution.

Other purification methods may be used to achieve the desired outcome. For example, WO 92/16484 (hereby incorporated by reference) discloses the use of gel permeation chromatography to isolate a fraction of poloxamer 188 that exhibits beneficial biological effects, without causing potentially deleterious side effects. The copolymer thus obtained had a polydispersity index of 1.07 or less, and was substantially saturated. The potentially harmful side effects were shown to be associated with the low molecular weight, unsaturated portion of the polymer, while the medically beneficial effects resided in the uniform higher molecular weight material. Other similarly improved copolymers were obtained by purifying either the polyoxypropylene center block during synthesis of the copolymer, or the copolymer product itself (e.g., U.S. Pat. No. 5,523,492 and U.S. Pat. No. 5,696,298; both of which are hereby incorporated by reference).

Further, a supercritical fluid extraction technique has been used to fractionate a polyoxyalkylene block copolymer as disclosed in U.S. Pat. No. 5,567,859 (hereby incorporated by reference). A purified fraction was obtained, which was composed of a fairly uniform polyoxyalkylene block copolymer having a polydispersity of less than 1.17. According to this method, the lower molecular weight fraction was removed in a stream of carbon dioxide maintained at a pressure of 2200 pounds per square inch (psi) and a temperature of 40° C.

Additionally, U.S. Pat. No. 5,800,711 (hereby incorporated by reference) discloses a process for the fractionation of polyoxyalkylene block copolymers by the batchwise removal of low molecular weight species using a salt extraction and liquid phase separation technique. Poloxamer 407 and poloxamer 188 were fractionated by this method. In each case, a copolymer fraction was obtained which had a higher average molecular weight and a lower polydispersity index as compared to the starting material. However, the changes in polydispersity index were modest and analysis by gel permeation chromatography indicated that some low-molecular-weight material remained. The viscosity of aqueous solutions of the fractionated polymers was significantly greater than the viscosity of the commercially available polymers at temperatures between 10° C. and 37° C., an important property for some medical and drug delivery applications. Nevertheless, some of the low molecular weight contaminants of these polymers are thought to cause deleterious side effects when used inside the body, making it especially important that they be removed in the fractionation process. As a consequence, polyoxyalkylene block copolymers fractionated by this process are not appropriate for all medical uses.

Previous work has shown that one can obtain cessation of intra-renal blood flow using a 22% solution of poloxamer 407, which forms a solid gel at 19° C. J. Raymond, A. Metcalfe, I. Salazkin, and A. Schwarz, “Temporary vascular occlusion with poloxamer 407,” Biomaterials 2004, 25, 3983. As mentioned above, the reverse thermosensitive polymer poloxamer 407 is a member of the poloxamer polymer family, which are well known water-soluble polymeric surfactants used in a variety of industrial and medical applications. However, this polymer was developed for a different purpose, namely hemostasis in smaller and cooler surface-exposed arteries, and for certain embodiments may be too low for injection into a solid organ as the injection force needed during the initial animal experiments was rather great (results not shown). Therefore, for certain embodiments, such as for use in intraparenchymal temporary hemostasis as applied to the kidney at normal body temperatures, the ideal reverse thermosensitive polymer may be different. In certain embodiments, a polymer with a higher transition temperature may be preferable. In certain embodiments, a polymer with a transition temperature of about 30° C. is preferred.

Modification of the transition temperature of a reverse thermosensitive polymer can be obtained in a number of ways. For example, the transition temperature can be modified either through the addition of transition temperature modifying additive or through the development of a modified polymer. The transition temperature can be influenced by a number of additives, e.g., the addition of pharmaceutical fatty acid excipients such as sodium oleate, sodium laurate or sodium caprate. Other possible pharmaceutical excipients may be solvents such as water, alcohols, especially C₁-C₅ alcohols such as ethanol, n-propanol, 2-propanol, isopropanol, t-butyl alcohol; ethers such as MTBE; ketones such as acetone, methyl ethyl ketone; humectants such as glycerol; glycols such as ethylene glycol, propylene glycol; emulsifiers such as lower, optionally polyhydric C₁-C₅ alcohols partially esterified with long-chain (C₁₂-C₂₄) fatty acids such as glycerol monostearate, isopropyl myristate, fatty acid ester of sugar alcohols such as sorbitan mono-fatty acid ester, polyethoxylated derivatives of such compounds, polyethoxyethylene fatty acid ester and fatty alcohol ether, cholesterol, cetyl stearyl alcohol, wool wax alcohols and synthetic surfactants with a low HLB value; solubilisers such as carbopol; low-viscosity paraffins, triglycerides; lipophilic substances such as isopropyl myristate; pH regulators such as TEA, carbonates and phosphates; chelating agents such as EDTA and salts thereof; as well as preservatives. Furthermore, the addition of other poloxamers to form mixtures of poloxamers is known to influence the transition temperature.

Another approach to achieving higher transition temperature is to use other poloxamers such as 288 and 188. There are no literature reports on the transition temperature of these poloxamers other than the statement that they are reverse thermosensitive. Table 2, FIG. 5, shows a variety of reverse thermosensitive polymer solutions and their gelation temperatures.

Approaches to increasing the transition temperature can be investigated by measuring the viscosity versus temperature curve of aqueous polymer solutions at various concentrations of the polymers and excipients. For certain embodiments, polymer solutions with increased transition temperature will be evaluated in vitro for the injection pressure required and the lowest injection pressure polymer solution will then be initially evaluated in vivo in a pig model of partial nephrectomy.

In certain embodiments, to aid in visualization, a contrast-enhancing agent can be added to the transient gel. Exemplarily contrast-enhancing agents are radiopaque materials, paramagnetic materials, heavy atoms, transition metals, lanthanides, actinides, dyes, and radionuclide-containing materials.

Selected Therapeutic Agents. The reversibly gelling polymers used in the methods of the invention have physico-chemical characteristics that make them suitable delivery vehicles for conventional small-molecule drugs, as well as macromolecular (e.g., peptides) drugs or other therapeutic products. Therefore, the composition comprising the thermosensitive polymer may further comprise a pharmaceutic agent selected to provide a pre-selected pharmaceutic effect. A pharmaceutic effect is one which seeks to prevent or treat the source or symptom of a disease or physical disorder. Pharmaceutics include those products subject to regulation under the FDA pharmaceutic guidelines. Importantly, the compositions used in methods of the invention are capable of solubilizing and releasing bioactive materials. Solubilization is expected to occur as a result of dissolution in the bulk aqueous phase or by incorporation of the solute in micelles created by the hydrophobic domains of the poloxamer. Release of the drug would occur through diffusion or network erosion mechanisms.

Those skilled in the art will appreciate that the compositions used in the methods of the invention may simultaneously be utilized to deliver a wide variety of pharmaceutics to a wound site. To prepare a pharmaceutic composition, an effective amount of pharmaceutically active agent(s), which imparts the desirable pharmaceutic effect is incorporated into the reversibly gelling composition used in the methods of the invention. Preferably, the selected agent is water soluble, which will readily lend itself to a homogeneous dispersion throughout the reversibly gelling composition. It is also preferred that the agent(s) is non-reactive with the composition. For materials, which are not water soluble, it is also within the scope of the methods of the invention to disperse or suspend lipophilic material throughout the composition. Myriad bioactive materials may be delivered using the methods of the present invention; the delivered bioactive material includes anesthetics, antimicrobial agents (antibacterial, antifungal, antiviral), anti-inflammatory agents, diagnostic agents, and wound-healing agents.

Because the reversibly gelling composition used in the methods of the present invention are suited for application under a variety of environmental conditions, a wide variety of pharmaceutically active agents may be incorporated into and administered via the composition. The pharmaceutic agent loaded into the polymer networks of the thermosensitive polymer may be any substance having biological activity, including proteins, polypeptides, polynucleotides, nucleoproteins, polysaccharides, glycoproteins, lipoproteins, and synthetic and biologically engineered analogs thereof.

A vast number of therapeutic agents may be incorporated in the polymers used in the methods of the present invention. In general, therapeutic agents which may be administered via the methods of the invention include, without limitation: antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins. Suitable pharmaceuticals for parenteral administration are well known as is exemplified by the Handbook on Injectable Drugs, 6th Edition, by Lawrence A. Trissel, American Society of Hospital Pharmacists, Bethesda, Md., 1990 (hereby incorporated by reference).

The pharmaceutically active compound may be any substance having biological activity, including proteins, polypeptides, polynucleotides, nucleoproteins, polysaccharides, glycoproteins, lipoproteins, and synthetic and biologically engineered analogs thereof. The term “protein” is art-recognized and for purposes of this invention also encompasses peptides. The proteins or peptides may be any biologically active protein or peptide, naturally occurring or synthetic.

Examples of proteins include antibodies, enzymes, growth hormone and growth hormone-releasing hormone, gonadotropin-releasing hormone, and its agonist and antagonist analogues, somatostatin and its analogues, gonadotropins such as luteinizing hormone and follicle-stimulating hormone, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. The pharmaceutical agents may be selected from insulin, antigens selected from the group consisting of MMR (mumps, measles and rubella) vaccine, typhoid vaccine, hepatitis A vaccine, hepatitis B vaccine, herpes simplex virus, bacterial toxoids, cholera toxin B-subunit, influenza vaccine virus, bordetela pertussis virus, vaccinia virus, adenovirus, canary pox, polio vaccine virus, plasmodium falciparum, Bacillus calmette geurin (BCG), Klebsiella pneumoniae, HIV envelop glycoproteins and cytokins and other agents selected from the group consisting of bovine somatropine (sometimes referred to as BST), estrogens, androgens, insulin growth factors (sometimes referred to as IGF), interleukin I, interleukin II and cytokins. Three such cytokins are interferon-β, interferon-γ and tuftsin.

Examples of bacterial toxoids that may be incorporated in the compositions used in the methods of the invention are tetanus, diphtheria, Pseudomonas A, Mycobaeterium tuberculosis. Examples of that may be incorporated in the compositions used in the occlusion methods of the invention are HIV envelope glycoproteins, e.g., gp 120 or gp 160, for AIDS vaccines. Examples of anti-ulcer H2 receptor antagonists that may be included are ranitidine, cimetidine and famotidine, and other anti-ulcer drugs are omparazide, cesupride and misoprostol. An example of a hypoglycaemic agent is glizipide.

Classes of pharmaceutically active compounds which can be loaded into that may be incorporated in the compositions used in the occlusion methods of the invention include, but are not limited to, anti-AIDS substances, anti-cancer substances, antibiotics, immunosuppressants (e.g., cyclosporine) anti-viral substances, enzyme inhibitors, neurotoxins, opioids, hypnotics, antihistamines, lubricants tranquilizers, anti-convulsants, muscle relaxants and anti-Parkinson substances, anti-spasmodics and muscle contractants, miotics and anti-cholinergics, anti-glaucoma compounds, anti-parasite and/or anti-protozoal compounds, anti-hypertensives, analgesics, anti-pyretics and anti-inflammatory agents such as NSAIDs, local anesthetics, ophthalmics, prostaglandins, anti-depressants, anti-psychotic substances, anti-emetics, imaging agents, specific targeting agents, neurotransmitters, proteins, cell response modifiers, and vaccines.

Exemplary pharmaceutical agents considered to be particularly suitable for incorporation in the compositions used in the methods of the invention include but are not limited to imidazoles, such as miconazole, econazole, terconazole, saperconazole, itraconazole, metronidazole, fluconazole, ketoconazole, and clotrimazole, luteinizing-hormone-releasing hormone (LHRH) and its analogues, nonoxynol-9, a GnRH agonist or antagonist, natural or synthetic progestrin, such as selected progesterone, 17-hydroxyprogeterone derivatives such as medroxyprogesterone acetate, and 19-nortestosterone analogues such as norethindrone, natural or synthetic estrogens, conjugated estrogens, estradiol, estropipate, and ethinyl estradiol, bisphosphonates including etidronate, alendronate, tiludronate, resedronate, clodronate, and pamidronate, calcitonin, parathyroid hormones, carbonic anhydrase inhibitor such as felbamate and dorzolamide, a mast cell stabilizer such as xesterbergsterol-A, lodoxamine, and cromolyn, a prostaglandin inhibitor such as diclofenac and ketorolac, a steroid such as prednisolone, dexamethasone, fluromethylone, rimexolone, and lotepednol, an antihistamine such as antazoline, pheniramine, and histiminase, pilocarpine nitrate, a beta-blocker such as levobunolol and timolol maleate. As will be understood by those skilled in the art, two or more pharmaceutical agents may be combined for specific effects. The necessary amounts of active ingredient can be determined by simple experimentation.

By way of example only, any of a number of antibiotics and antimicrobials may be included in the thermosensitive polymers used in the methods of the invention. Antimicrobial drugs preferred for inclusion in compositions used in the occlusion methods of the invention include salts of lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole and amanfadine and the like.

By way of example only, in the case of anti-inflammation, non-steroidal anti-inflammatory agents (NSAIDS) may be incorporated in the compositions used in the occlusion methods of the invention, such as propionic acid derivatives, acetic acid, fenamic acid derivatives, biphenylcarboxylic acid derivatives, oxicams, including but not limited to aspirin, acetaminophen, ibuprofen, naproxen, benoxaprofen, flurbiprofen, fenbufen, ketoprofen, indoprofen, pirprofen, carporfen, and bucloxic acid and the like.

Injection System. A delivery system may be used to facilitate and control injection of the reverse thermosensitive polymer composition. Ideally, the injection system would minimize the need for dissection of the artery prior to injection. Further, in constructing an optimal injection system it may be helpful to determine the thumb pressure required to inject the polymer in liquid form through various diameter needles while maintaining a flow rate of 0.5 mL per second. A tensile testing apparatus (e.g., Instron®) can be used measure the force needed and resulting rate of compression to depress the plunger.

In certain embodiments, use of a cannula that can be detected in a vessel using standard non-invasive systems in the operating room (e.g., a handheld ultrasound) will aid in verifying that the cannula is correctly placed in the renal artery. The catheter may be a dilatation catheter. In one embodiment, the catheter is 3-10 French in size, and more preferably 3-6 French. In another embodiment, a catheter can be used to dispense one or more fluids other than, or in addition to, the polymer solution. In said embodiment the catheter may be a multiple lumen catheter with one lumen for the delivery of the polymer solution, other lumen for the delivery of other fluids such as a contrast agent solution.

In another embodiment, the syringe or other mechanism may be used to inject the polymer solution into the body can be, for example, a 1-100 cc syringe, a 1-50 cc syringe or a 1-5 cc. Pressure applied to the syringe can be applied by hand or by an automated syringe pusher. In certain embodiments, a system to provide auxiliary power to a syringe for injection of a viscous material (e.g., a spring loaded plunger assisted device) may be used.

Kits. This invention also provides kits for conveniently and effectively implementing the methods of this invention. Such kits comprise any of the polymers of the present invention or a combination thereof, and a means for facilitating their use consistent with methods of this invention. Such kits may also included ice, a cold pack, or other means of cooling. Such kits provide a convenient and effective means for assuring that the methods are practiced in an effective manner. The compliance means of such kits includes any means which facilitates practicing a method of this invention. Such compliance means include instructions, packaging, and dispensing means, and combinations thereof. Kit components may be packaged for either manual or partially or wholly automated practice of the foregoing methods. In other embodiments, this invention contemplates a kit including block copolymers of the present invention, and optionally instructions for their use. In certain embodiments, the reverse thermosensitive copolymers of such a kit of the present invention are contained in one or more syringes.

Transient Gels of the Invention. One aspect of the invention relates to a transient gel.

In certain embodiments, the present invention relates to the aforementioned transient gel and any of the attendant limitations, wherein the transient gel is a gel at mammalian physiological temperature.

In certain embodiments, the present invention relates to any one of the aforementioned transient gels and any of the attendant limitations, wherein said transient gel comprises at least one optionally purified reverse thermosensitive polymer.

In certain embodiments, the present invention relates to any one of the aforementioned transient gels and any of the attendant limitations, wherein said transient gel comprises about 5% to about 35% of said reverse thermosensitive polymer.

In certain embodiments, the present invention relates to any one of the aforementioned transient gels and any of the attendant limitations, wherein said transient gel comprises about 10% to about 30% of said reverse thermosensitive polymer.

In certain embodiments, the present invention relates to any one of the aforementioned transient gels and any of the attendant limitations, wherein said at least one optionally purified reverse thermosensitive polymer has a polydispersity index from about 1.5 to about 1.0.

In certain embodiments, the present invention relates to any one of the aforementioned transient gels and any of the attendant limitations, wherein said at least one optionally purified reverse thermosensitive polymer has a polydispersity index from about 1.2 to about 1.0.

In certain embodiments, the present invention relates to any one of the aforementioned transient gels and any of the attendant limitations, wherein said at least one optionally purified reverse thermosensitive polymer is selected from the group consisting of block copolymers, random copolymers, graft polymers, and branched copolymers.

In certain embodiments, the present invention relates to any one of the aforementioned transient gels and any of the attendant limitations, wherein said at least one optionally purified reverse thermosensitive polymer is a polyoxyalkylene block copolymer.

In certain embodiments, the present invention relates to any one of the aforementioned transient gels and any of the attendant limitations, wherein said at least one optionally purified reverse thermosensitive polymer is selected from the group consisting of poloxamers and poloxamines.

In certain embodiments, the present invention relates to any one of the aforementioned transient gels and any of the attendant limitations, wherein said at least one optionally purified reverse thermosensitive polymer is selected from the group consisting of poloxamer 407, poloxamer 288, poloxamer 188, poloxamer 338, poloxamer 118, Tetronic® 1107 or Tetronic® 1307.

In certain embodiments, the present invention relates to any one of the aforementioned transient gels and any of the attendant limitations, wherein said at least one optionally purified reverse thermosensitive polymer is poloxamer 407.

In certain embodiments, the present invention relates to any one of the aforementioned transient gels and any of the attendant limitations, wherein said at least one optionally purified reverse thermosensitive polymer is selected from the group consisting of purified poloxamers and purified poloxamines.

In certain embodiments, the present invention relates to any one of the aforementioned transient gels and any of the attendant limitations, wherein said at least one optionally purified reverse thermosensitive polymer is selected from the group consisting of purified poloxamer 407, purified poloxamer 288, purified poloxamer 188, purified poloxamer 338, purified poloxamer 118, purified Tetronic® 1107 or purified Tetronic® 1307.

In certain embodiments, the present invention relates to any one of the aforementioned transient gels and any of the attendant limitations, wherein said at least one optionally purified reverse thermosensitive polymer is purified poloxamer 407.

In certain embodiments, the present invention relates to any one of the aforementioned transient gels and any of the attendant limitations, wherein said transient gel comprises an excipient.

In certain embodiments, the present invention relates to any one of the aforementioned transient gels and any of the attendant limitations, wherein said transient gel comprises a pharmaceutical fatty acid excipient.

In certain embodiments, the present invention relates to any one of the aforementioned transient gels and any of the attendant limitations, wherein said pharmaceutical fatty acid excipient is sodium oleate, sodium laurate or sodium caprate.

In certain embodiments, the present invention relates to any one of the aforementioned transient gels and any of the attendant limitations, wherein said transient gel comprises a therapeutic agent.

In certain embodiments, the present invention relates to any one of the aforementioned transient gels and any of the attendant limitations, wherein the therapeutic agent is selected from the group consisting of antiinflammatories, antibiotics, antimicrobials, chemotherapeutics, antivirals, analgesics, and antiproliferatives.

In certain embodiments, the present invention relates to any one of the aforementioned transient gels and any of the attendant limitations, wherein the therapeutic agent is an antibiotic.

In certain embodiments, the present invention relates to the aforementioned method, wherein said transient gel comprises a contrast-enhancing agent.

In certain embodiments, the present invention relates to the aforementioned method, wherein said contrast-enhancing agent is selected from the group consisting of radiopaque materials, paramagnetic materials, heavy atoms, transition metals, lanthanides, actinides, dyes, and radionuclide-containing materials.

In certain embodiments, the present invention relates to any one of the aforementioned transient gels and any of the attendant limitations, wherein said transient gel has a transition temperature of between about 20° C. and about 50° C.

In certain embodiments, the present invention relates to any one of the aforementioned transient gels and any of the attendant limitations, wherein said transient gel has a transition temperature of between about 30° C. and about 40° C.

In certain embodiments, the present invention relates to any one of the aforementioned transient gels and any of the attendant limitations, wherein the volume of said transient gel at physiological temperature is about 80% to about 120% of its volume below its transition temperature.

In certain embodiments, the present invention relates to any one of the aforementioned transient gels and any of the attendant limitations, wherein the volume of said transient gel at physiological temperature is about 80% to about 120% of its volume below its transition temperature; and said transient gel has a transition temperature of between about 20° C. and about 50° C.

In certain embodiments, the present invention relates to any one of the aforementioned transient gels and any of the attendant limitations, wherein the volume of said transient gel at physiological temperature is about 80% to about 120% of its volume below its transition temperature; and said transient gel has a transition temperature of between about 30° C. and about 40° C.

In certain embodiments, the present invention relates to any one of the aforementioned transient gels and any of the attendant limitations, wherein the volume of said transient gel at physiological temperature is about 80% to about 120% of its volume below its transition temperature; said transient gel has a transition temperature of between about 20° C. and about 50° C.; and said transient gel comprises at least one optionally purified reverse thermosensitive polymer selected from the group consisting of poloxamers and poloxamines.

In certain embodiments, the present invention relates to any one of the aforementioned transient gels and any of the attendant limitations, wherein the volume of said transient gel at physiological temperature is about 80% to about 120% of its volume below its transition temperature; said transient gel has a transition temperature of between about 30° C. and about 40° C.; and said transient gel comprises at least one optionally purified reverse thermosensitive polymer selected from the group consisting of poloxamers and poloxamines.

In certain embodiments, the present invention relates to any one of the aforementioned transient gels and any of the attendant limitations, wherein said transient gel comprises an anionic, cationic, or non-ionically crosslinkable polymer.

In certain embodiments, the present invention relates to any one of the aforementioned transient gels and any of the attendant limitations, wherein said transient gel comprises a polymer selected from the group consisting of alginic acid, sodium alginate, potassium alginate, sodium gellan, potassium gellan, carboxy methyl cellulose, hyaluronic acid and polyvinyl alcohol.

In certain embodiments, the present invention relates to any one of the aforementioned transient gels and any of the attendant limitations, wherein said transient gel comprises phosphate, citrate, borate, succinate, maleate, adipate, oxalate, calcium, magnesium, barium, strontium, or a combination thereof.

In certain embodiments, the present invention relates to any one of the aforementioned transient gels and any of the attendant limitations, wherein said transient gel comprises a polymer selected from the group consisting of alginic acid, sodium alginate, potassium alginate, sodium gellan and potassium gellan; and further comprises calcium, magnesium or barium.

In certain embodiments, the present invention relates to any one of the aforementioned transient gels and any of the attendant limitations, wherein said transient gel comprises a polymer selected from the group consisting of alginic acid, sodium alginate or potassium alginate; and further comprises composition comprises calcium.

In certain embodiments, the present invention relates to any one of the aforementioned transient gels and any of the attendant limitations, wherein said transient gel comprises a polymer selected from the group consisting of sodium gellan and potassium gellan; and further comprises magnesium.

In certain embodiments, the present invention relates to any one of the aforementioned transient gels and any of the attendant limitations, wherein said transient gel comprises hyaluronic acid; and further comprises calcium.

In certain embodiments, the present invention relates to any one of the aforementioned transient gels and any of the attendant limitations, wherein said transient gel comprises polyvinyl alcohol; and further comprises borate.

In certain embodiments, the present invention relates to any one of the aforementioned transient gels and any of the attendant limitations, wherein said transient gel comprises proteins selected from the group consisting of collagen, gelatin, elastin, albumin, protamine, fibrin, fibrinogen, keratin, reelin, caseine, and mixture thereof.

In certain embodiments, the present invention relates to any one of the aforementioned transient gels and any of the attendant limitations, wherein said transient gel comprises hyaluronic acid, chitosan, or a mixture thereof.

In certain embodiments, the present invention relates to any one of the aforementioned transient gels and any of the attendant limitations, wherein said transient gel comprises synthetic materials selected from the group consisting of alginate, pectin, methylcellulose, carboxymethylcellulose, and mixtures thereof.

In certain embodiments, the present invention relates to any one of the aforementioned transient gels and any of the attendant limitations, wherein said transient gel comprises crosslinkable polymers.

Methods of the Invention. One aspect of the invention relates to a method of perfusive organ hemostasis in a subject, comprising the step of introducing into an arterial vessel in fluid communication with an organ a volume of a composition, wherein said volume is sufficient to perfuse substantially said organ; and said composition forms a transient gel in said organ.

In certain embodiments, the present invention relates to any one of the aforementioned methods and any of the attendant limitations, wherein the volume of said composition is about 1-25 mL or about 1-10 mL.

In certain embodiments, the present invention relates to any one of the aforementioned methods and any of the attendant limitations, wherein said composition is introduced over about 1-30 seconds or about 2-20 seconds.

In certain embodiments, the present invention relates to any one of the aforementioned methods and any of the attendant limitations, wherein said transient gel is a gel at mammalian physiological temperature.

In certain embodiments, the present invention relates to any one of the aforementioned methods and any of the attendant limitations, wherein said transient gel comprises at least one optionally purified reverse thermosensitive polymer.

In certain embodiments, the present invention relates to any one of the aforementioned methods and any of the attendant limitations, wherein said transient gel comprises about 5% to about 35% of said reverse thermosensitive polymer.

In certain embodiments, the present invention relates to any one of the aforementioned methods and any of the attendant limitations, wherein said transient gel comprises about 10% to about 30% of said reverse thermosensitive polymer.

In certain embodiments, the present invention relates to any one of the aforementioned methods and any of the attendant limitations, wherein said at least one optionally purified reverse thermosensitive polymer has a polydispersity index from about 1.5 to about 1.0.

In certain embodiments, the present invention relates to any one of the aforementioned methods and any of the attendant limitations, wherein said at least one optionally purified reverse thermosensitive polymer has a polydispersity index from about 1.2 to about 1.0.

In certain embodiments, the present invention relates to any one of the aforementioned methods and any of the attendant limitations, wherein said at least one optionally purified reverse thermosensitive polymer is selected from the group consisting of block copolymers, random copolymers, graft polymers, and branched copolymers.

In certain embodiments, the present invention relates to any one of the aforementioned methods and any of the attendant limitations, wherein said at least one optionally purified reverse thermosensitive polymer is a polyoxyalkylene block copolymer.

In certain embodiments, the present invention relates to any one of the aforementioned methods and any of the attendant limitations, wherein said at least one optionally purified reverse thermosensitive polymer is selected from the group consisting of poloxamers and poloxamines.

In certain embodiments, the present invention relates to any one of the aforementioned methods and any of the attendant limitations, wherein said at least one optionally purified reverse thermosensitive polymer is selected from the group consisting of poloxamer 407, poloxamer 288, poloxamer 188, poloxamer 338, poloxamer 118, Tetronic® 1107 and Tetronic® 1307.

In certain embodiments, the present invention relates to any one of the aforementioned methods and any of the attendant limitations, wherein said at least one optionally purified reverse thermosensitive polymer is poloxamer 407.

In certain embodiments, the present invention relates to any one of the aforementioned methods and any of the attendant limitations, wherein said at least one optionally purified reverse thermosensitive polymer is selected from the group consisting of purified poloxamers and purified poloxamines.

In certain embodiments, the present invention relates to any one of the aforementioned methods and any of the attendant limitations, wherein said at least one optionally purified reverse thermosensitive polymer is selected from the group consisting of purified poloxamer 407, purified poloxamer 288, purified poloxamer 188, purified poloxamer 338, purified poloxamer 118, purified Tetronic® 1107 and purified Tetronic® 1307.

In certain embodiments, the present invention relates to any one of the aforementioned methods and any of the attendant limitations, wherein said at least one optionally purified reverse thermosensitive polymer is purified poloxamer 407.

In certain embodiments, the present invention relates to any one of the aforementioned methods and any of the attendant limitations, wherein said transient gel comprises an excipient.

In certain embodiments, the present invention relates to any one of the aforementioned methods and any of the attendant limitations, wherein said transient gel comprises a pharmaceutical fatty acid excipient.

In certain embodiments, the present invention relates to any one of the aforementioned methods and any of the attendant limitations, wherein said pharmaceutical fatty acid excipient is sodium oleate, sodium laurate or sodium caprate.

In certain embodiments, the present invention relates to any one of the aforementioned methods and any of the attendant limitations, wherein said transient gel comprises a therapeutic agent.

In certain embodiments, the present invention relates to any one of the aforementioned methods and any of the attendant limitations, wherein the therapeutic agent is selected from the group consisting of antiinflammatories, antibiotics, antimicrobials, chemotherapeutics, antivirals, analgesics, and antiproliferatives.

In certain embodiments, the present invention relates to any one of the aforementioned methods and any of the attendant limitations, wherein the therapeutic agent is an antibiotic.

In certain embodiments, the present invention relates to the aforementioned method, wherein said transient gel comprises a contrast-enhancing agent.

In certain embodiments, the present invention relates to the aforementioned method, wherein said contrast-enhancing agent is selected from the group consisting of radiopaque materials, paramagnetic materials, heavy atoms, transition metals, lanthanides, actinides, dyes, and radionuclide-containing materials.

In certain embodiments, the present invention relates to any one of the aforementioned methods and any of the attendant limitations, wherein said transient gel has a transition temperature of between about 20° C. and about 50° C.

In certain embodiments, the present invention relates to any one of the aforementioned methods and any of the attendant limitations, wherein said transient gel has a transition temperature of between about 30° C. and about 40° C.

In certain embodiments, the present invention relates to any one of the aforementioned methods and any of the attendant limitations, wherein the volume of said transient gel at physiological temperature is about 80% to about 120% of its volume below its transition temperature.

In certain embodiments, the present invention relates to any one of the aforementioned methods and any of the attendant limitations, wherein the volume of said transient gel at physiological temperature is about 80% to about 120% of its volume below its transition temperature; and said transient gel has a transition temperature of between about 20° C. and about 50° C.

In certain embodiments, the present invention relates to any one of the aforementioned methods and any of the attendant limitations, wherein the volume of said transient gel at physiological temperature is about 80% to about 120% of its volume below its transition temperature; and said transient gel has a transition temperature of between about 30° C. and about 40° C.

In certain embodiments, the present invention relates to any one of the aforementioned methods and any of the attendant limitations, wherein the volume of said transient gel at physiological temperature is about 80% to about 120% of its volume below its transition temperature; said transient gel has a transition temperature of between about 20° C. and about 50° C.; and said transient gel comprises at least one optionally purified reverse thermosensitive polymer selected from the group consisting of poloxamers and poloxamines.

In certain embodiments, the present invention relates to any one of the aforementioned methods and any of the attendant limitations, wherein the volume of said transient gel at physiological temperature is about 80% to about 120% of its volume below its transition temperature; said transient gel has a transition temperature of between about 30° C. and about 40° C.; and said transient gel comprises at least one optionally purified reverse thermosensitive polymer selected from the group consisting of poloxamers and poloxamines.

In certain embodiments, the present invention relates to any one of the aforementioned methods and any of the attendant limitations, wherein said transient gel comprises an anionic, cationic, or non-ionically crosslinkable polymer.

In certain embodiments, the present invention relates to any one of the aforementioned methods and any of the attendant limitations, wherein said transient gel comprises a polymer selected from the group consisting of alginic acid, sodium alginate, potassium alginate, sodium gellan, potassium gellan, carboxy methyl cellulose, hyaluronic acid and polyvinyl alcohol.

In certain embodiments, the present invention relates to any one of the aforementioned methods and any of the attendant limitations, wherein said transient gel comprises phosphate, citrate, borate, succinate, maleate, adipate, oxalate, calcium, magnesium, barium, or strontium.

In certain embodiments, the present invention relates to any one of the aforementioned methods and any of the attendant limitations, wherein said transient gel comprises a polymer selected from the group consisting of alginic acid, sodium alginate, potassium alginate, sodium gellan and potassium gellan; and calcium, magnesium or barium.

In certain embodiments, the present invention relates to any one of the aforementioned methods and any of the attendant limitations, wherein said transient gel comprises a polymer selected from the group consisting of alginic acid, sodium alginate and potassium alginate; and calcium.

In certain embodiments, the present invention relates to any one of the aforementioned methods and any of the attendant limitations, wherein said transient gel comprises a polymer selected from the group consisting of sodium gellan and potassium gellan; and magnesium.

In certain embodiments, the present invention relates to any one of the aforementioned methods and any of the attendant limitations, wherein said transient gel comprises hyaluronic acid; and calcium.

In certain embodiments, the present invention relates to any one of the aforementioned methods and any of the attendant limitations, wherein said transient gel comprises polyvinyl alcohol; and borate.

In certain embodiments, the present invention relates to any one of the aforementioned methods and any of the attendant limitations, wherein said transient gel comprises a protein selected from the group consisting of collagen, gelatin, elastin, albumin, protamine, fibrin, fibrinogen, keratin, reelin, and caseine.

In certain embodiments, the present invention relates to any one of the aforementioned methods and any of the attendant limitations, wherein said transient gel comprises hyaluronic acid, or chitosan.

In certain embodiments, the present invention relates to any one of the aforementioned methods and any of the attendant limitations, wherein said transient gel comprises alginate, pectin, methylcellulose, or carboxymethylcellulose.

In certain embodiments, the present invention relates to any one of the aforementioned methods and any of the attendant limitations, wherein said transient gel comprises a crosslinkable polymer.

In certain embodiments, the present invention relates to any one of the aforementioned methods and any of the attendant limitations, wherein said organ is a highly vascularized organ.

In certain embodiments, the present invention relates to any one of the aforementioned methods and any of the attendant limitations, wherein said organ is a kidney, a liver, a prostate, a brain, a uterus, or a spleen.

In certain embodiments, the present invention relates to any one of the aforementioned methods and any of the attendant limitations, wherein said organ is a kidney, a liver or a prostate.

In certain embodiments, the present invention relates to any one of the aforementioned methods and any of the attendant limitations, wherein said organ is a kidney.

In certain embodiments, the present invention relates to any one of the aforementioned methods and any of the attendant limitations, wherein the lifetime of said transient gel is about twenty minutes.

In certain embodiments, the present invention relates to any one of the aforementioned methods and any of the attendant limitations, wherein the lifetime of said transient gel is about thirty minutes.

In certain embodiments, the present invention relates to any one of the aforementioned methods and any of the attendant limitations, wherein the lifetime of said transient gel is about forty minutes.

In certain embodiments, the present invention relates to any one of the aforementioned methods and any of the attendant limitations, wherein said subject is a mammal.

In certain embodiments, the present invention relates to any one of the aforementioned methods and any of the attendant limitations, wherein said subject is a human.

In certain embodiments, the present invention relates to any one of the aforementioned methods and any of the attendant limitations, wherein said composition is introduced using a syringe, cannula, catheter or percutaneous access device.

In certain embodiments, the present invention relates to any one of the aforementioned methods and any of the attendant limitations, wherein said composition is introduced using a dual lumen catheter or a triple lumen catheter.

In certain embodiments, the present invention relates to any one of the aforementioned methods and any of the attendant limitations, wherein the catheter is 3-10 French or 3-6 French in size.

In certain embodiments, the present invention relates to any one of the aforementioned methods and any of the attendant limitations, wherein the catheter can be used to dispense one or more fluids other than, or in addition to, the polymer solution. For example, the catheter may be a multiple lumen catheter with one lumen for the delivery of the polymer solution, other lumen for the delivery of other fluids such as a contrast agent solution.

In certain embodiments, the present invention relates to any one of the aforementioned methods and any of the attendant limitations, wherein said composition is introduced using a syringe.

In certain embodiments, the present invention relates to any one of the aforementioned methods and any of the attendant limitations, wherein the syringe used to inject the polymer solution into the body can be a 1-100 cc syringe, a 1-50 cc syringe or a 1-5 cc syringe. Pressure applied to the syringe can be applied by hand or by an automated syringe pusher.

In certain embodiments, the present invention relates to any one of the aforementioned methods and any of the attendant limitations, wherein said composition is cooled to about 15° C. prior to introduction.

In certain embodiments, the present invention relates to any one of the aforementioned methods and any of the attendant limitations, wherein said composition is cooled to about 10° C. prior to introduction.

In certain embodiments, the present invention relates to any one of the aforementioned methods and any of the attendant limitations, wherein said composition is cooled to about 5° C. prior to introduction.

In certain embodiments, the present invention relates to any one of the aforementioned methods and any of the attendant limitations, wherein said composition is cooled to about 0° C. prior to introduction.

In certain embodiments, the present invention relates to any one of the aforementioned methods and any of the attendant limitations, wherein said composition is cooled with ice, water, or a cold pack prior to introduction.

In certain embodiments, the present invention relates to any one of the aforementioned methods and any of the attendant limitations, further comprising introducing saline to aid in the disolution of said transient gel.

In certain embodiments, the present invention relates to any one of the aforementioned methods and any of the attendant limitations, further comprising the step of cooling said organ.

Kits of the Inventions. In certain embodiments, the present invention relates to a kit for perfusive organ hemostasis, comprising instructions for use thereof; and a first container comprising a volume of a composition, wherein said composition forms a transient gel at mammalian physiological temperature.

In certain embodiments, the present invention relates to the aforementioned kit and any of the attendant limitations, further comprising a cold pack.

In certain embodiments, the present invention relates to the aforementioned kit and any of the attendant limitations, further comprising a syringe or cannula.

In certain embodiments, the present invention relates to the aforementioned kit and any of the attendant limitations, wherein said composition comprises at least one optionally purified reverse thermosensitive polymer.

In certain embodiments, the present invention relates to the aforementioned kit and any of the attendant limitations, wherein said composition comprises about 5% to about 35% of said reverse thermosensitive polymer.

In certain embodiments, the present invention relates to the aforementioned kit and any of the attendant limitations, wherein said composition comprises about 10% to about 30% of said reverse thermosensitive polymer.

In certain embodiments, the present invention relates to the aforementioned kit and any of the attendant limitations, wherein said at least one optionally purified reverse thermosensitive polymer has a polydispersity index from about 1.5 to about 1.0.

In certain embodiments, the present invention relates to the aforementioned kit and any of the attendant limitations, wherein said at least one optionally purified reverse thermosensitive polymer has a polydispersity index from about 1.2 to about 1.0.

In certain embodiments, the present invention relates to the aforementioned kit and any of the attendant limitations, wherein said at least one optionally purified reverse thermosensitive polymer is selected from the group consisting of block copolymers, random copolymers, graft polymers, and branched copolymers.

In certain embodiments, the present invention relates to the aforementioned kit and any of the attendant limitations, wherein said at least one optionally purified reverse thermosensitive polymer is a polyoxyalkylene block copolymer.

In certain embodiments, the present invention relates to the aforementioned kit and any of the attendant limitations, wherein said at least one optionally purified reverse thermosensitive polymer is selected from the group consisting of poloxamers and poloxamines.

In certain embodiments, the present invention relates to the aforementioned kit and any of the attendant limitations, wherein said at least one optionally purified reverse thermosensitive polymer is selected from the group consisting of poloxamer 407, poloxamer 288, poloxamer 188, poloxamer 338, poloxamer 118, Tetronic® 1107 and Tetronic® 1307.

In certain embodiments, the present invention relates to the aforementioned kit and any of the attendant limitations, wherein said at least one optionally purified reverse thermosensitive polymer is poloxamer 407.

In certain embodiments, the present invention relates to the aforementioned kit and any of the attendant limitations, wherein said at least one optionally purified reverse thermosensitive polymer is selected from the group consisting of purified poloxamers and purified poloxamines.

In certain embodiments, the present invention relates to the aforementioned kit and any of the attendant limitations, wherein said at least one optionally purified reverse thermosensitive polymer is selected from the group consisting of purified poloxamer 407, purified poloxamer 288, purified poloxamer 188, purified poloxamer 338, purified poloxamer 118, purified Tetronic® 1107 and purified Tetronic® 1307.

In certain embodiments, the present invention relates to the aforementioned kit and any of the attendant limitations, wherein said at least one optionally purified reverse thermosensitive polymer is purified poloxamer 407.

In certain embodiments, the present invention relates to the aforementioned kit and any of the attendant limitations, wherein said composition comprises an excipient.

In certain embodiments, the present invention relates to the aforementioned kit and any of the attendant limitations, wherein said composition comprises a pharmaceutical fatty acid excipient.

In certain embodiments, the present invention relates to the aforementioned kit and any of the attendant limitations, wherein said pharmaceutical fatty acid excipient is sodium oleate, sodium laurate or sodium caprate.

In certain embodiments, the present invention relates to the aforementioned kit and any of the attendant limitations, wherein said composition comprises a therapeutic agent.

In certain embodiments, the present invention relates to the aforementioned kit and any of the attendant limitations, wherein the therapeutic agent is selected from the group consisting of antiinflammatories, antibiotics, antimicrobials, chemotherapeutics, antivirals, analgesics, and antiproliferatives.

In certain embodiments, the present invention relates to the aforementioned kit and any of the attendant limitations, wherein the therapeutic agent is an antibiotic.

In certain embodiments, the present invention relates to the aforementioned kit and any of the attendant limitations, wherein said composition comprises a contrast-enhancing agent.

In certain embodiments, the present invention relates to the aforementioned kit and any of the attendant limitations, wherein said contrast-enhancing agent is selected from the group consisting of radiopaque materials, paramagnetic materials, heavy atoms, transition metals, lanthanides, actinides, dyes, and radionuclide-containing materials.

In certain embodiments, the present invention relates to the aforementioned kit and any of the attendant limitations, wherein said composition has a transition temperature of between about 20° C. and about 50° C.

In certain embodiments, the present invention relates to the aforementioned kit and any of the attendant limitations, wherein said composition has a transition temperature of between about 30° C. and about 40° C.

In certain embodiments, the present invention relates to the aforementioned kit and any of the attendant limitations, wherein the volume of said composition at physiological temperature is about 80% to about 120% of its volume below its transition temperature.

In certain embodiments, the present invention relates to the aforementioned kit and any of the attendant limitations, wherein the volume of said composition at physiological temperature is about 80% to about 120% of its volume below its transition temperature; and said composition has a transition temperature of between about 20° C. and about 50° C.

In certain embodiments, the present invention relates to the aforementioned kit and any of the attendant limitations, wherein the volume of said composition at physiological temperature is about 80% to about 120% of its volume below its transition temperature; and said composition has a transition temperature of between about 30° C. and about 40° C.

In certain embodiments, the present invention relates to the aforementioned kit and any of the attendant limitations, wherein the volume of said composition at physiological temperature is about 80% to about 120% of its volume below its transition temperature; said composition has a transition temperature of between about 20° C. and about 50° C.; and said composition comprises at least one optionally purified reverse thermosensitive polymer selected from the group consisting of poloxamers and poloxamines.

In certain embodiments, the present invention relates to the aforementioned kit and any of the attendant limitations, wherein the volume of said composition at physiological temperature is about 80% to about 120% of its volume below its transition temperature; said composition has a transition temperature of between about 30° C. and about 40° C.; and said composition comprises at least one optionally purified reverse thermosensitive polymer selected from the group consisting of poloxamers and poloxamines.

In certain embodiments, the present invention relates to the aforementioned kit and any of the attendant limitations, wherein said composition comprises an anionic, cationic, or non-ionically crosslinkable polymer.

In certain embodiments, the present invention relates to the aforementioned kit and any of the attendant limitations, wherein said composition comprises a polymer selected from the group consisting of alginic acid, sodium alginate, potassium alginate, sodium gellan, potassium gellan, carboxy methyl cellulose, hyaluronic acid and polyvinyl alcohol.

In certain embodiments, the present invention relates to the aforementioned kit and any of the attendant limitations, wherein said composition comprises phosphate, citrate, borate, succinate, maleate, adipate, oxalate, calcium, magnesium, barium, or strontium.

In certain embodiments, the present invention relates to the aforementioned kit and any of the attendant limitations, wherein said composition comprises a polymer selected from the group consisting of alginic acid, sodium alginate, potassium alginate, sodium gellan and potassium gellan; and calcium, magnesium or barium.

In certain embodiments, the present invention relates to the aforementioned kit and any of the attendant limitations, wherein said composition comprises a polymer selected from the group consisting of alginic acid, sodium alginate or potassium alginate; and calcium.

In certain embodiments, the present invention relates to the aforementioned kit and any of the attendant limitations, wherein said composition comprises a polymer selected from the group consisting of sodium gellan and potassium gellan; and magnesium.

In certain embodiments, the present invention relates to the aforementioned kit and any of the attendant limitations, wherein said composition comprises hyaluronic acid; and calcium.

In certain embodiments, the present invention relates to the aforementioned kit and any of the attendant limitations, wherein said composition comprises polyvinyl alcohol; and borate.

In certain embodiments, the present invention relates to the aforementioned kit and any of the attendant limitations, wherein said composition comprises a protein selected from the group consisting of collagen, gelatin, elastin, albumin, protamine, fibrin, fibrinogen, keratin, reelin, and caseine.

In certain embodiments, the present invention relates to the aforementioned kit and any of the attendant limitations, wherein said composition comprises hyaluronic acid, or chitosan.

In certain embodiments, the present invention relates to the aforementioned kit and any of the attendant limitations, wherein said composition comprises alginate, pectin, methylcellulose, or carboxymethylcellulose.

In certain embodiments, the present invention relates to the aforementioned kit and any of the attendant limitations, wherein said composition comprises a crosslinkable polymer.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Partial Nephrectomy

Partial nephrectomy with internal renal vascular occlusion using a reverse thermosensitive polymer solution was attempted in 2 pigs. After the pig was intubated and sedated a right flank incision was performed from the rib cage to just above the pubic symphysis. Full exposure of the right kidney, the renal vein, renal artery, aorta and vena cava was obtained via a retroperitoneal approach. The aorta was canulated retrograde from the right iliac artery with the catheter tip lying about 15 mm proximal to the origin of the right renal artery. Twelve ml of the transient gel (20% poloxamer 407) was injected into the aorta, which was not sufficient to occlude the aorta, or the renal artery, but which lead to complete cessation of blood flow within the kidney itself. Fifteen minutes later, the kidney still had no circulation, and the lower pole was resected with no trace of bleeding. The resected end of the kidney was then oversewn and the kidney was cooled externally.

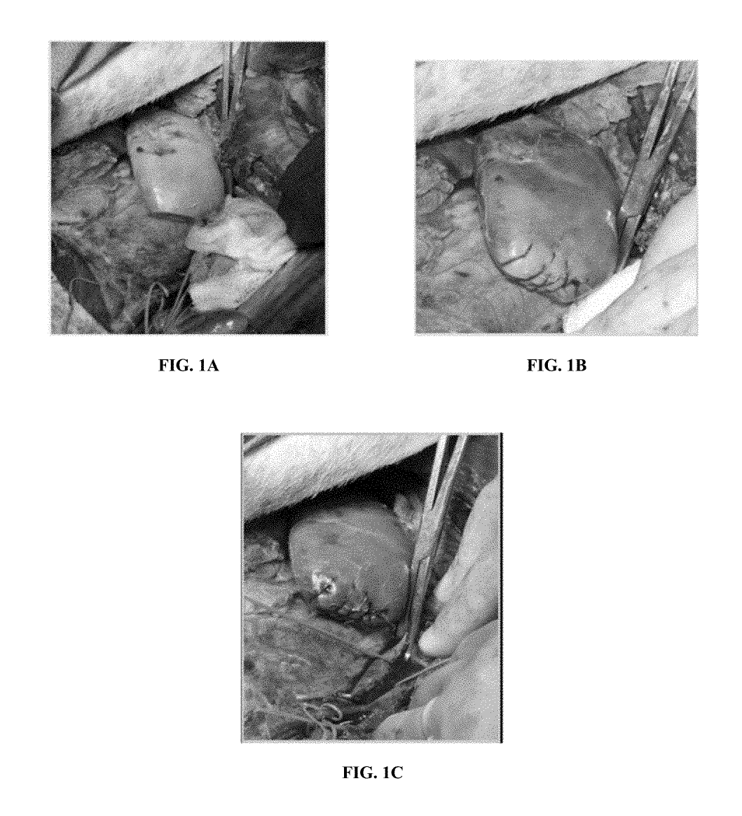

Blood flow returned thereafter and the kidney resumed its normal appearance, including a normal pulse in the right renal artery. There was no bleeding from the nephrectomy site and the renal surface remained bloodless in both pigs. The overall time for the partial nephrectomy was less than 10 minutes as there was no need to take care of bleeding during the surgery. See FIG. 1.

### Example 2

### Renal Exposure

A study of renal exposure was similar to the above procedure. A catheter was inserted directly into the renal artery. A slow injection of 1.5 ml of the polymer produced ischemia identical to that described above. A heminephrectomy was performed in this case. Again, the surgery was largely bloodless, but after transecting a major renal artery branch in the pelvis of the kidney, a slight oozing of the polymer gel, followed by bleeding, was noted. This was readily and easily oversewn, allowing easy completion of the heminephrectomy and closure of the remaining kidney as described above. The remaining half kidney also resumed normal appearance and normal histology after transient cooling allowed re-liquefaction of the polymer. On microscopic examination in this case, no pathology was noted in either the resected or remaining half of the kidney.

### Example 3

### Sample Purification

Poloxamer 407 (486.0 g, lot number WPHT-543B), purchased from BASF Corporation, Mount Olive, N.J., was dissolved in deionized water (15,733 g). The solution was maintained at 0.1° C. and 2335.1 g of (NH₄)₂SO₄ were added. The solution was equilibrated at 2° C. and after two distinct phases formed, the lower phase was discarded, and the upper phase (2060 g) was collected and weighed. Deionized water (14159 g) was added and the solution was equilibrated to 2° C. Next, 2171.6 g of (NH₄)₂SO₄ were added with stirring. After the salt was dissolved, the solution was maintained at approximately 2° C. until two phases formed. The upper phase (3340 g) was isolated and diluted with 12879 g of deionized water. The solution was chilled to about 2.2° C. and 2062 g of (NH₄)₂SO₄ were added. The phases were allowed to separate as above. The upper phase was isolated and extracted with 4 liters of dichloromethane. Two phases were allowed to form overnight. The organic (lower) phase was isolated and approximately 2 kg of sodium sulfate (Na₂SO₄) were added to it to remove the remaining water. The dichloromethane phase was filtered through a PTFE filter (0.45 μm pore size) to remove the undissolved salts. The dichloromethane was removed under vacuum at approximately 30° C. Final traces of dichloromethane were removed by drying in an oven overnight at about 30° C. A total of 297.6 g of fractionated poloxamer 407 (lot number 00115001) were recovered. The chemical and physical characteristics of the fractionated poloxamer 407 are compared to those of the starting material in FIG. 5, Table 1.

### Example 4

### Sample In-Vitro Viscosity Testing

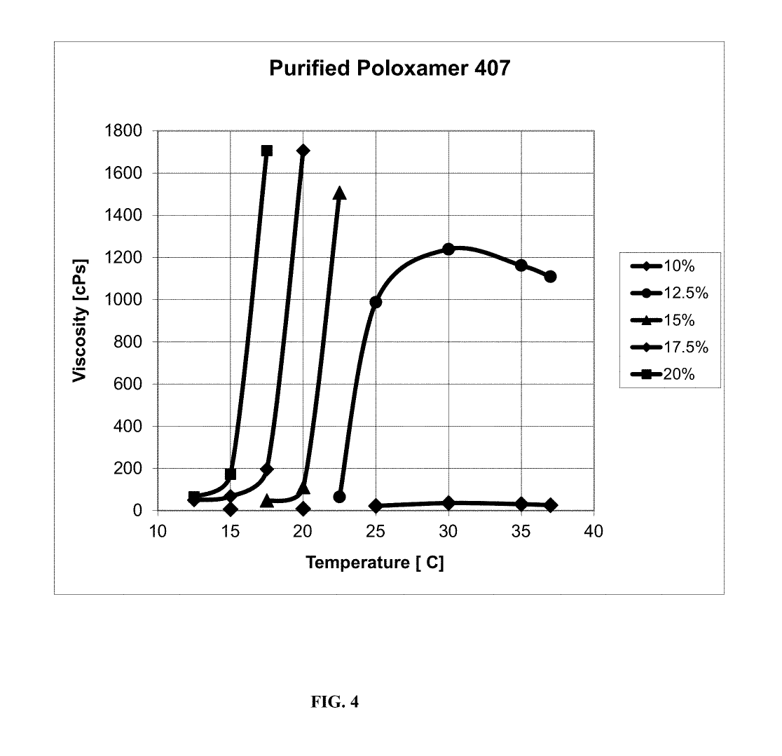

Viscosity changes can be measured in a Brookfield Cone and Cup viscometer with temperature control. For example, a graph of the viscosity changes of poloxamer 407 (FIG. 4) clearly shows polymer concentrations from approximately 12.5 w % until at least 20 w % will show steep increases in solution viscosities with temperature. The onset of gelation is dependent on the temperature and higher polymer concentrations lead to earlier onsets of gelation. Furthermore, polymer concentrations below approximately 12.5 w % do not demonstrate an increase in solution viscosity with temperature and remain liquid even at body temperature.

These two findings demonstrate the potential operation principle purified reverse thermosensitive polymers. The polymer solution can be injected as a soft gel at a specific temperature (e.g., the temperature of a typical OR) into the arteriotomy and the rise in temperature leads to a stiff gel. The gel will start to dissolve in blood and when the concentration of the polymer decreases, without any possibility of resolidifying into a gel at physiological temperatures. Alternatively, cooling of the gel with ice or cold saline would liquefy the gel as the temperature falls below the gelation point. As a liquid, it quickly dilutes in blood and again there is no possibility for it to turn back into a gel at physiological temperatures.

### Example 5

### Gelation Temperature of Selected Pluronic® and Tetronic® Polymer Solutions

The polymer was weighed into a plastic tube. To achieve the required concentration the weight was multiplied by 4, for 25 weight percent (w %), and by 5, for 20 weight percent (w %), and the required final weight was achieved by adding saline. The solutions were placed in the fridge at 4° C. and usually were ready within 24 hours. Gelation points were measured in a Brookfield viscometer and the point at which viscosity exceeded the range of the plate/cone (greater than about 102,000 cP) was called the gelation temperature. See FIG. 5, Table 2.

### Example 6a

### Studies on Vertebrate Animals

Animal studies could further support the feasibility of using reverse thermosensitive polymers to achieve effective hemostasis during partial nephrectomy. This involves all aspects of the arterial and venous system of the kidney, as well as the detailed anatomy and physiology of the medulla, which can not be replicated in an in-vitro model. The anatomy of the omnivore swine is similar to that of the human, thereby creating the best simulation of conditions in a human operation, and the swine kidney is a classic model for human kidneys. M. M. Swindle, et al., “Swine as models in experimental surgery,” J Invest Surg. 1988, 1(1), 65-79.

Acute experiments using ordinary farm swine will be performed. The pigs will weigh about 30 to 40 kg, slightly smaller than human size but sufficient to evaluate the feasibility of our hemostatic agent. Each pig will undergo a partial nephrectomy. The use of the transient gels of the invention to obtain temporary hemostasis will be evaluated.

The pig will be induced with an intramuscular mixture containing acepromazine (1.1 mg/kg) and atropine (0.05 mg/kg) as a pre-anesthetic. Five to fifteen minutes after pre-anesthetic administration the pig will be induced with ketamine (20 mg/kg) and xylazine (2.0 mg/kg) via IM injection. After the pig has reached a level of anesthesia to allow endotracheal intubation it will be intubated. Following intubation, the animal will be maintained throughout the preparation and surgery period on inhalation anesthesia (on a semiclosed circuit inhalation of isoflurane to effect). Assisted ventilation, if necessary, will be accomplished with a ventilator. An IV catheter will be aseptically placed in the ear vein or other appropriate vessel.

Following induction of anesthesia, the pig will be prepared by shaving the skin, and brought to the operating room. The animal will be ventilated as above. A right paramedian incision from the costal margin to just above the inguinal ligament will be opened to the peritoneum by dividing the obligue abdominal muscles about one inch lateral to their rectus insertion. The peritoneum will be reflected from the kidney, exposing the vena cava and the aorta. The kidney, renal artery and renal vein will be cleaned and exposed. A thermister will be place in the aorta just proximal to the renal artery for monitoring temperature of the blood at the time of injection.

To induce temporary renal ischemia, a 25 gague catheter will be introduced to the renal artery by puncture, and advanced 1.5 cm distally. It will remain in place for the duration of the experiment. Approximately 1-2 cc of the polymer will be injected. The volume and rate of injection will be determined prior to surgery based, in part, on viscosity measurements and polymerization temperature information. Data regarding each injection will include: injection time, rate of injection, volume of injection, blood temperature, time to cessation of blood flow, completeness of blood flow cessation, time to perform partial nephrectomy, including plication of the severed renal surface, and time to return of blood flow. These data will be correlated to the physical properties of the polymer.

The animal will be maintained until there is complete return of blood flow and normal appearance of the kidney. It will then be kept in stable condition for one additional hour, to ensure complete reperfusion of the remaining renal mass. The animal will then be euthanized with an overdose of pentobarbital and phenytoin. The resected renal portion and the re-perfused renal portion will be sent for microscopic analysis. The remaining pig carcass will be disposed of. Euthanasia will be achieved with an intravenous injection of pentobarbital sodium and phenytoin as follows: Drugs: pentobarbital sodium and phenytoin sodium; Dose: 1 cc/10 pounds; and Route: rapid intravenous injection. If the animal is in a deep plane of anesthesia a saturated solution of KCl may be administered intravenously to accomplish euthanasia. Following administration of the drug, the animal will be examined to ensure that respiratory function has ceased and there is no palpable cardiac function.

### Example 6b

### Sample Procedure for Pig Anesthesia

The following are sample protocols which can be followed in performing the procedures described in Example 6a.

Animal Identification: Each animal will be identified by a tattoo in the pinna of the ear or an ear tag.

Anesthesia; option #1: The pig will be induced with an intramuscular cocktail containing Telazol (4.4 mg/kg), Xylazine (2.2 mg/kg) and Atropine (0.05 mg/kg) to a level of anesthesia to allow endotracheal intubation. Following intubation, the animal will be maintained throughout the preparation and surgery period on inhalation anesthesia (on a semi-closed circuit inhalation of isoflurane to effect). Assisted ventilation, if necessary, will be accomplished with a ventilator. An IV catheter will be aseptically placed in the ear vein or other appropriate vessel.

Anesthesia; option #2: The pig will be induced with an intramuscular cocktail containing acepromazine (1.1 mg/kg), atropine (0.05 mg/kg) as a preanesthetic. Five to fifteen minutes after preanesthetic administration the pig will be induced with ketamine (20 mg/kg) and xylazine (2.0 mg/kg) via IM injection. After the pig has reached a level of anesthesia to allow endotracheal intubation it will be intubated. Following intubation, the animal will be maintained throughout the preparation and surgery period on inhalation anesthesia (on a semiclosed circuit inhalation of isoflurane to effect). Assisted ventilation, if necessary, will be accomplished with a ventilator. An IV catheter will be aseptically placed in the ear vein or other appropriate vessel.

Surgical Preparation: Sterile procedures will be followed in the event of survival surgery. Depilitation of the surgical site will be accomplished with an electric animal clipper equipped with a surgical shaving blade. The area will be vacuumed to remove all clippings and debris, then scrubbed in an alternating sequence with an aqueous iodophor solution of 1% available iodine and 70% isopropyl alcohol a minimum of three times. Following drying, the entire area will be painted with a solution of 0.7% available iodine and 74% isopropyl alcohol. The anesthetized and surgically prepared animal will be delivered to the operating table and placed in the desired recumbent position. A sterile surgical drape will be placed over the entire animal and surgical table. Intravenous fluid therapy will be administered at the maintenance rate of 4-6 ml/kg/hr.

Clinical Observations: Temperature, pulse and respiratory rate will be monitored postoperatively as directed by the veterinary staff and/or primary investigator. The animal will be observed daily postoperatively to determine health status on the basis of food consumption, excretion and general attitude. All animals will also be observed daily for the presence of pain and/or discomfort and analgesics administered as necessary.

Analgesics: During the first 48 hrs. postoperative period, buprenex (0.01-0.02 mg/kg/IM Q 12 hr) will be administered. Following the first 48 hr. period, analgesics will be administered as necessary.

Euthanasia: Euthanasia will be achieved with an intravenous injection of pentobarbital sodium and phenytoin sodium as directed by the label instructions on the bottle. If the animal is in a deep plane of anesthesia, a saturated solution of potassium chloride may be administered intravenous to accomplish euthanasia. The potassium ion is cardiotoxic, and rapid intravenous or intracardiac administration of 1-2 mmol/kg of body weight will cause cardiac arrest. Following administration of the drug, the animal will be examined to ensure that respiratory function has ceased and there is no palpable cardiac function.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of perfusive organ hemostasis in partial nephrectomy in a subject, comprising the step of introducing into an arterial vessel in fluid communication with a kidney a volume of a composition, wherein said volume is sufficient to perfuse substantially said kidney; and said composition: (a) comprises at least one optionally purified reverse thermosensitive polymer selected from the group consisting of the group consisting of poloxamer 407, poloxamer 288, poloxamer 188, poloxamer 338, poloxamer 118, Tetronic® 1107 and Tetronic® 1307; and (b) forms a transient gel in said kidney.

2. The method of claim 1, wherein the volume of said composition is about 1-25 mL.

3. The method of claim 1, wherein said composition is introduced over about 1-30 seconds.

4. The method of claim 1, wherein said composition is a gel at mammalian physiological temperature.

5. (canceled)

6. The method of claim 1, wherein said composition comprises about 5% to about 35% of said reverse thermosensitive polymer.

7. (canceled)

8. The method of claim 1, wherein said at least one optionally purified reverse thermosensitive polymer has a polydispersity index from about 1.5 to about 1.0.

9-16. (canceled)

17. The method of claim 1, wherein said at least one optionally purified reverse thermosensitive polymer is purified poloxamer 407.

18. The method of claim 1, wherein said composition comprises an excipient.

19. The method of claim 1, wherein said composition comprises a pharmaceutical fatty acid excipient.

20. The method of claim 19, wherein said pharmaceutical fatty acid excipient is sodium oleate, sodium laurate or sodium caprate.

21. The method of claim 1, wherein said composition comprises a therapeutic agent.

22. The method of claim 21, wherein the therapeutic agent is selected from the group consisting of antiinflammatories, antibiotics, antimicrobials, chemotherapeutics, antivirals, analgesics, and antiproliferatives.

23. The method of claim 21, wherein the therapeutic agent is an antibiotic.

24. The method of claim 1, wherein said transient gel composition comprises a contrast-enhancing agent.

25. The method of claim 24, wherein said contrast-enhancing agent is selected from the group consisting of radiopaque materials, paramagnetic materials, heavy atoms, transition metals, lanthanides, actinides, dyes, and radionuclide-containing materials.

26-40. (canceled)

41. The method of a claim 1, wherein said composition has a transition temperature of between about 20° C. and about 50° C.

42. The method of a claim 1, wherein the volume of said composition at physiological temperature is about 80% to about 120% of its volume below its transition temperature.

43. The method of a claim 1, wherein the lifetime of said transient gel is about twenty minutes.

44. The method of a claim 1, wherein said composition is cooled to about 15° C. prior to introduction.

45. The method of a claim 1, wherein saline is further introduced to aid in the dissolution of said transient gel.
